(19) United States
(12) Patent Application Publication
(10) Pub. No.: US 2018/0339274 A1
(43) Pub. Date: Nov. 29, 2018

(54) GAS SEPARATION MEMBRANE, GAS SEPARATION MODULE, GAS SEPARATOR, GAS SEPARATION METHOD, AND POLYIMIDE COMPOUND

(71) Applicant: FUJIFILM Corporation, Tokyo (JP)

(72) Inventors: Keisuke KODAMA, Kanagawa (JP); Motoi HARADA, Kanagawa (JP)

(73) Assignee: FUJIFILM Corporation, Tokyo (JP)

(21) Appl. No.: 16/045,769

(22) Filed: Jul. 26, 2018

Related U.S. Application Data
(63) Continuation of application No. PCT/JP2017/004252, filed on Feb. 6, 2017.

(30) Foreign Application Priority Data
Feb. 26, 2016 (JP) 2016-036426

(51) Int. Cl. B01D 71/64; B01D 53/22; B01D 69/12; B01D 69/10; C08G 73/10; C01B 32/50; C07C 7/144

(52) U.S. Cl. CPC B01D 71/64; B01D 53/228; B01D 69/12; B01D 69/10; C01B 2210/0012; C01B 32/50; C07C 7/144; C01B 2210/007; C08G 73/1067

(57) ABSTRACT

A gas separation membrane includes a gas separation layer that contains the polyimide compound having the structural portion represented by Formula (1). A gas separation module includes the gas separation membrane, a gas separator includes the gas separation module, and a gas separation method is performed using the gas separation membrane.

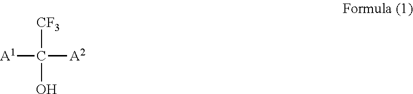

A¹ and A² represent a linking site, a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group. Here, at least one of A¹ or A² represents a linking site.

### CROSS-REFERENCE TO RELATED APPLICATIONS

This application is a Continuation of PCT International Application No. PCT/JP2017/4252, filed on Feb. 6, 2017, which claims priority under 35 U.S.C. § 119(a) to Japanese Patent Application No. 2016-036426, filed on Feb. 26, 2016. Each of the above application(s) is hereby expressly incorporated by reference, in its entirety, into the present application.

### BACKGROUND OF THE INVENTION

### 1. Field of the Invention

The present invention relates to a gas separation membrane, a gas separation module, a gas separator, a gas separation method, and a polyimide compound.

### 2. Description of the Related Art

A material formed of a polymer compound has a gas permeability specific to the material. Based on this property, it is possible to cause selective permeation and separation out of a target gas component using a membrane formed of a specific polymer compound. As an industrial application for this gas separation membrane related to the problem of global warming, separation and recovery of carbon dioxide from large-scale carbon dioxide sources using this gas separation membrane has been examined in thermal power plants, cement plants, or ironworks blast furnaces. Further, this membrane separation technique has been attracting attention as means for solving environmental issues with relatively little energy. A membrane separation method used as means for removing impurities such as carbon dioxide from natural gas or biogas (gas generated due to fermentation or anaerobic digestion, for example, biological excrement, organic fertilizers, biodegradable substances, sewage, garbage, or energy crops) mainly containing methane and carbon dioxide may be exemplified.

In purification of natural gas using a membrane separation method, excellent gas permeability and gas separation selectivity are required in order to more efficiently separate gas. Various membrane materials have been examined for the purpose of realizing excellent gas permeability and gas separation selectivity, and a gas separation membrane obtained by using a polyimide compound has been examined as part of examination of membrane materials.

For example, JP2013-10096A describes that polyimide containing a hydroxy-1,1,1,3,3,3-hexafluoroisopropyl group in a repeating unit thereof has excellent solubility in an organic solvent or excellent formability, and a gas separation membrane obtained by using such polyimide has excellent gas separation performance. Further, JP2011-183370A describes that a gas separation membrane that contains aromatic polyimide formed of a specific repeating unit obtained by using ditrifluoromethyl diamino diphenyl ethers has excellent gas separation performance and excellent mechanical properties.

### SUMMARY OF THE INVENTION

In order to obtain a practical gas separation membrane, it is necessary to ensure sufficient gas permeability and to realize improved gas separation selectivity. However, gas permeability and gas separation selectivity have a so-called trade-off relationship. Therefore, by adjusting a copolymerization component of a polyimide compound used for a gas separation layer, any of the gas permeability and the gas separation selectivity of the gas separation layer can be improved, but it is considered to be difficult to achieve both properties at high levels.

Further, in an actual plant, a membrane is plasticized due to the influence of impurity components (such as benzene, toluene, and xylene) present in natural gas and this results in a problem of degradation in gas separation selectivity. Accordingly, a gas separation membrane is also required to improve the gas permeability and the gas separation selectivity and to have plasticity resistance that enables high gas permeability and gas separation selectivity to be maintained even in the presence of the impurity components.

A polyimide compound is known to exhibit the gas separation performance as described above. However, a polyimide compound typically has degraded plasticity resistance, and the gas separation performance thereof is likely to be degraded in the coexistence of impurity components such as toluene. Particularly in a case where a polyimide compound having a high gas permeability is used for a gas separation layer, the gas separation layer is easily affected by the impurity components, and thus swelling of the gas separation layer is promoted. Therefore, in the gas separation layer obtained by using a polyimide compound, it is difficult to achieve both of the gas permeability and the plasticity resistance at high levels.

An object of the present invention is to provide a gas separation membrane which is capable of achieving both of excellent gas permeability and excellent gas separation selectivity even in a case of being used under a high pressure condition, includes a gas separation layer that is unlikely to be swollen even in a case of being brought into contact with impurity components such as toluene, and has excellent plasticity resistance. Further, an object of the present invention is to provide a gas separation module, a gas separator, and a gas separation method obtained by using the gas separation membrane. Further, an object of the present invention is to provide a polyimide compound suitable as a gas separation layer of the gas separation membrane.

As the result of intensive examination repeatedly conducted by the present inventors in consideration of the above-described problems, it was found that, in a case where a structure containing a trifluoromethyl group and a hydroxy group as substituents on the same carbon is introduced into the structure of a polyimide compound and such a polyimide compound is used for a gas separation layer of the gas separation membrane, this gas separation membrane exhibits excellent gas permeability and excellent gas separation selectivity, is unlikely to be affected by impurity components such as toluene, and has excellent plasticity resistance. The present invention has been completed after repeated examination based on these findings.

The above-described objects are achieved by the following means.

[1] A gas separation membrane comprising: a gas separation layer which contains a polyimide compound having a structural portion represented by Formula (1),

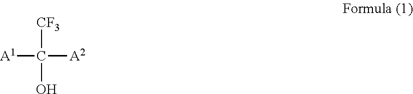

in Formula (1), A¹ and A² represent a linking site, a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, provided that at least one of A¹ or A² represents a linking site.

[2] The gas separation membrane according to [1], in which the polyimide compound has a unit structure represented by Formula (2),

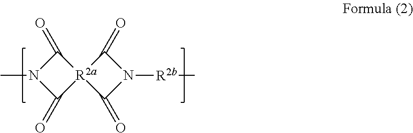

in Formula (2), R^{2a} represents a tetravalent linking group, and R^{2b} represents a divalent linking group, provided that at least one of R^{2a} or R^{2b} has the structural portion represented by Formula (1).

[3] The gas separation membrane according to [2], in which R^{2a} in Formula (2) has the structural portion represented by Formula (1), and R^{2a} is represented by any of Formulae (3-1) to (3-3),

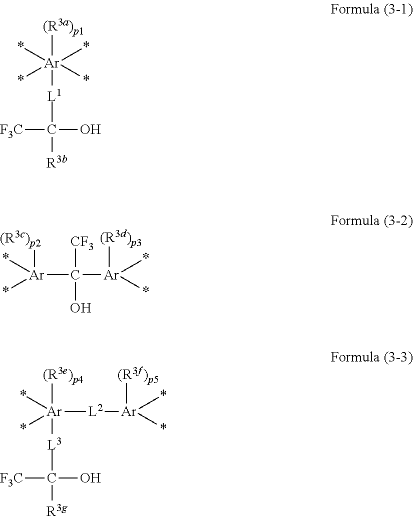

in Formulae (3-1) to (3-3), Ar represents an aromatic ring, the symbol “*” represents a linking site, L¹, L², and L³ represent a single bond or a divalent linking group, R^{3a}, R^{3c}, R^{3d}, R^{3e}, and R^{3f} represent a substituent, R^{3b} and R^{3g} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, and p1 to p5 represent an integer of 0 to 20.

[4] The gas separation membrane according to [2] or [3], in which R^{2b} in Formula (2) has the structural portion represented by Formula (1), and R^{2b} is represented by any of Formulae (4-1) to (4-3),

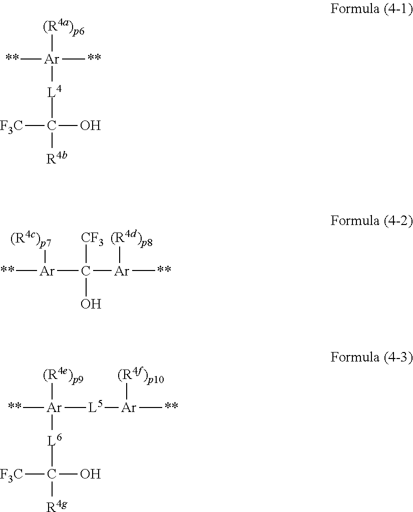

in Formulae (4-1) to (4-3), Ar represents an aromatic ring, the symbol “**” represents a linking site, L⁴, L⁵, and L⁶ represent a single bond or a divalent linking group, R^{4a}, R^{4c}, R^{4d}, R^{4e}, and R^{4f} represent a substituent, R^{4b} and R^{4g} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, and p6 to p10 represent an integer of 0 to 20.

[5] The gas separation membrane according to [2], in which R^{2a} in Formula (2) is represented by any of Formulae (I-1) to (I-28),

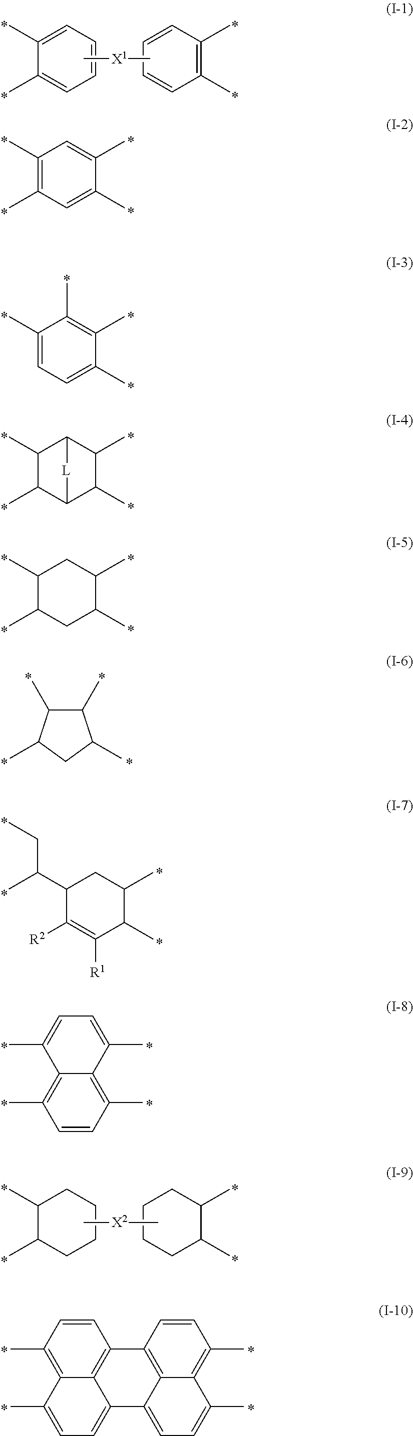

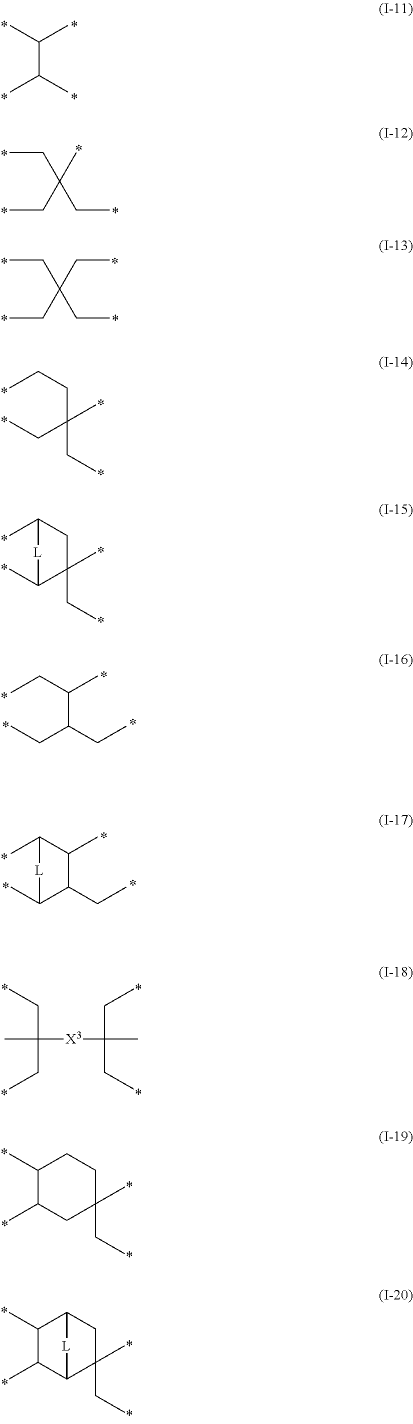

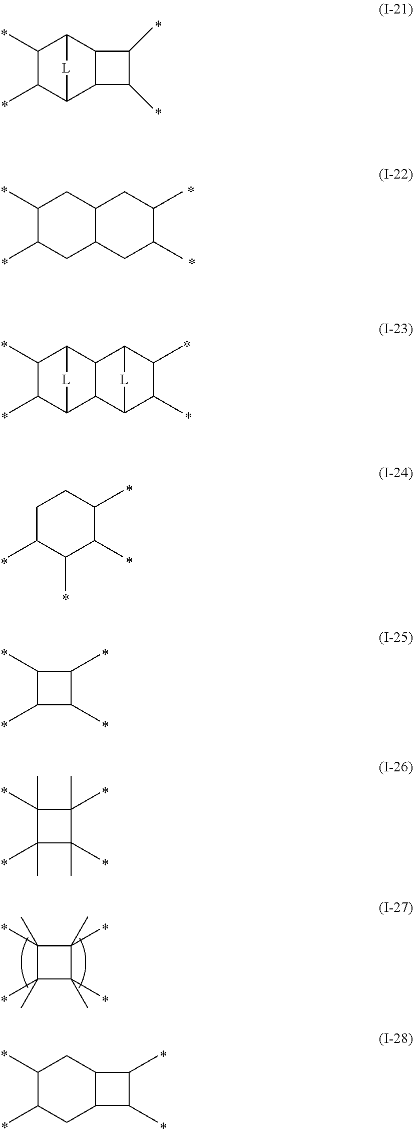

in Formulae (I-1) to (I-28), X¹ to X³ represent a single bond or a divalent linking group, L represents —CH═CH— or —CH₂—, R¹ and R² represent a hydrogen atom or a substituent that does not have a structural portion represented by Formula (1), and the symbol “*” represents a linking site.

[6] The gas separation membrane according to [2] or [3], in which R^{2b} in Formula (2) is represented by Formula (II-a) or (II-b),

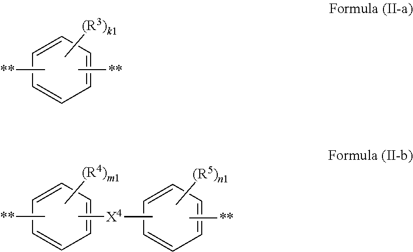

in Formula (II-a), R³ represents a substituent which does not have the structural portion represented by Formula (1), and k1 represents an integer of 0 to 4,

in Formula (II-b), R⁴ and R⁵ represent a substituent which does not have the structural portion represented by Formula (1) or a group that are linked to each other to form a ring together with X⁴, m1 and n1 represent an integer of 0 to 4, and X⁴ represents a single bond or a divalent linking group, and

the symbol “**” represents a linking site.

[7] The gas separation membrane according to any one of [1] to [6], in which the content of the structural portion represented by Formula (1) in the polyimide compound is 0.50 mmol/g or greater.

[8] The gas separation membrane according to any one of [1] to [7], in which a toluene swelling ratio of the polyimide compound is 35% or less.

[9] The gas separation membrane according to any one of [1] to [8], in which the gas separation membrane is a gas separation composite membrane which includes a gas permeating support layer and the gas separation layer.

[10] A gas separation module comprising: the gas separation membrane according to any one of [1] to [9].

[11] A gas separator comprising: the gas separation module according to [10].

[12] A gas separation method which is performed using the gas separation membrane according to any one of [1] to [9].

[13] A polyimide compound which is represented by any of Formulae (5) to (7),

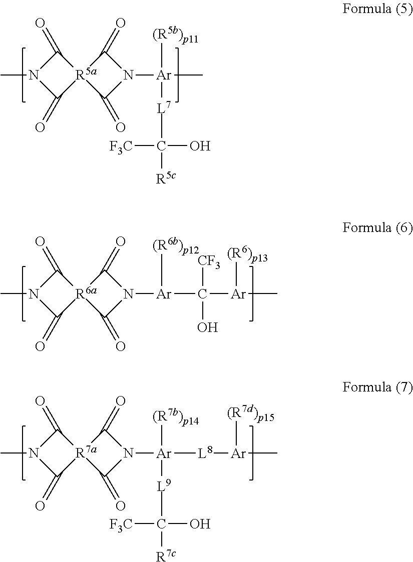

in Formulae (5) to (7), Ar represents an aromatic ring, R^{5a}, R^{6a}, and R^{7a} represent a tetravalent linking group, R^{5b}, R^{6b}, R^{6c}, R^{7b}, and R^{7d} represent a substituent, R^{5C} and R^{7c} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, L⁷, L⁸, and L⁹ represent a single bond or a divalent linking group, and p11 to p15 represent an integer of 0 to 20.

[14] A polyimide compound which is represented by any of Formulae (8) to (10),

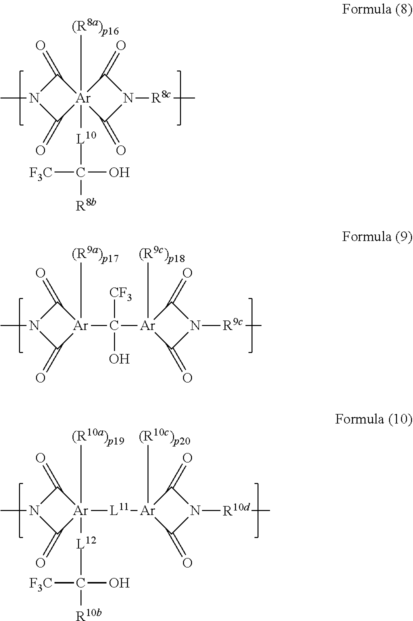

in Formulae (8) to (10), Ar represents an aromatic ring, R^{8a}, R^{9a}, R^{9b}, R^{10a}, and R^{10c} represent a substituent, R^{8b} and R^{10b} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, R^{8c}, R^{9c}, and R^{10d} represent a divalent linking group, L¹⁰, L¹¹, and L¹² represent a single bond or a divalent linking group, and p16 to p20 represent an integer of 0 to 20.

In the present specification, in a case where a plurality of substituents or linking groups (hereinafter, referred to as substituents or the like) shown by specific symbols are present or a plurality of substituents are defined simultaneously or alternatively, this means that the respective substituents may be the same as or different from each other. The same applies to the definition of the number of substituents or the like. Moreover, in a case where there is a repetition of a plurality of partial structures shown by means of the same display in the formula, the respective partial structures or repeating units may be the same as or different from each other. Further, in a case where a plurality of substituents and the like are adjacent to one another, this means that these may be linked to each other or condensed to form a ring unless otherwise specified.

In regard to compounds or groups described in the present specification, the description includes salts thereof and ions thereof in addition to the compounds or the groups. Further, the description includes those obtained by changing a part of the structure thereof within the range in which the effects of the purpose are exhibited.

A substituent (the same applies to a linking group) in which substitution or unsubstitution is not specified in the present specification may include an optional substituent of the group within a range in which desired effects are exhibited. The same applies to a compound in which substitution or unsubstitution is not specified.

A preferable range of a substituent group Z described below is set as a preferable range of a substituent in the present specification unless otherwise specified.

The gas separation membrane, the gas separation module, and the gas separator of the present invention enable achievement both of excellent gas permeability and excellent gas separation selectivity and enable gas separation with a high speed and high selectivity even in a case of being used under a high pressure condition. Further, the gas separation membrane, the gas separation module, and the gas separator of the present invention are unlikely to be swollen even in a case where the gas separation layer is brought into contact with impurity components such as toluene and have excellent plasticity resistance.

By using the polyimide compound of the present invention for the gas separation layer of the gas separation membrane, it is possible to provide a gas separation membrane which is capable of achieving both of excellent gas permeability and excellent gas separation selectivity at high levels even in a case of being used under a high pressure condition and has excellent plasticity resistance.

According to the gas separation method of the present invention, it is possible to achieve both of excellent gas permeability and excellent gas separation selectivity at high levels even in a case of being used under a high pressure condition. Further, it is possible to continuously exhibit excellent gas permeability and excellent gas separation selectivity even at the time of separating gas that contains impurity components (plasticizing components) such as toluene.

### BRIEF DESCRIPTION OF THE DRAWINGS

FIG. 1 is a cross-sectional view schematically illustrating an embodiment of a gas separation composite membrane according to the present invention.

FIG. 2 is a cross-sectional view schematically illustrating another embodiment of a gas separation composite membrane according to the present invention.

FIG. 3 shows ¹H-NMR spectral data of a diamine 1 synthesized in an example.

FIG. 4 shows ¹H-NMR spectral data of a polyimide compound (P-101) synthesized in an example.

### DESCRIPTION OF THE PREFERRED EMBODIMENTS

Hereinafter, preferred embodiments of the present invention will be described.

A gas separation membrane of the present invention contains a specific polyimide compound in a gas separation layer.

[Polyimide Compound]

The polyimide compound used in the present invention has a structural portion represented by Formula (1).

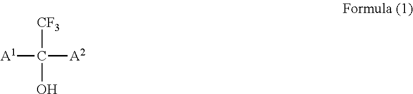

In Formula (1), A¹ and A² represent a linking site, a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group. Here, at least one of A¹ or A² represents a linking site.

Here, the expression “A¹ or A² represents a linking site” means that the structural portion represented by Formula (1) is incorporated in the polyimide compound through such a linking site. In other words, the structural portion represented by Formula (1) is present as a substituent in the polyimide compound in a case where only one of A¹ and A² represents a linking site and the structural portion represented by Formula (1) is incorporated in the polyimide compound as a divalent linking group in a case where both of A¹ and A² represent a linking site.

Examples of the halogen atom which can be employed as A¹ and A² include a fluorine atom, a chlorine atom, a bromine atom, and an iodine atom. Among these, a fluorine atom or a chlorine atom is preferable.

The number of carbon atoms of the carbamoyl group which can be employed as A¹ and A² is preferably in a range of 1 to 10, more preferably in a range of 1 to 6, and still more preferably in a range of 1 to 4. Further, an unsubstituted carbamoyl group is particularly preferable as the carbamoyl group. In a case where the carbamoyl group includes a substituent, an alkyl group is preferable as such a substituent.

The number of carbon atoms of the acyl group which can be employed as A¹ and A² is preferably in a range of 2 to 10, more preferably in a range of 2 to 6, still more preferably in a range of 2 to 4, and particularly preferably 2 or 3. Further, an alkylcarbonyl group is preferable as this acyl group.

The number of carbon atoms of the acyloxy group which can be employed as A¹ and A² is preferably in a range of 2 to 10, more preferably in a range of 2 to 6, still more preferably in a range of 2 to 4, and particularly preferably 2 or 3. Further, an alkylcarbonyloxy group is preferable as this acyloxy group.

The number of carbon atoms of the sulfamoyl group which can be employed as A¹ and A² is preferably in a range of 0 to 10, more preferably in a range of 0 to 6, and still more preferably in a range of 0 to 4. Further, an unsubstituted sulfamoyl group is particularly preferable as the sulfamoyl group. In a case where the sulfamoyl group includes a substituent, an alkyl group is preferable as such a substituent.

An alkyl group constituting the alkylsulfinyl group which can be employed as A¹ and A² may be linear or branched. The number of carbon atoms of this alkylsulfinyl group is preferably in a range of 1 to 10, more preferably in a range of 1 to 6, still more preferably in a range of 1 to 4, and particularly preferably in a range of 1 to 3.

The number of carbon atoms of an aryl group constituting the arylsulfinyl group which can be employed as A¹ and A² is preferably in a range of 6 to 20, more preferably in a range of 6 to 15, and still more preferably in a range of 6 to 12. Further, a phenyl group is even still more preferable as the arylsulfinyl group. Such a phenyl group may include a substituent, and a halogen atom (preferably a fluorine atom), a hydroxy group, a carboxy group, a sulfo group, or a sulfamoyl group (preferably an unsubstituted sulfamoyl group) is preferable as such a substituent.

An alkyl group constituting the alkylsulfonyloxy group which can be employed as A¹ and A² may be linear or branched. The number of carbon atoms of this alkylsulfonyloxy group is preferably in a range of 1 to 10, more preferably in a range of 1 to 6, still more preferably in a range of 1 to 4, and particularly preferably in a range of 1 to 3.

An alkyl group constituting the alkoxycarbonyl group which can be employed as A¹ and A² may be linear or branched. The number of carbon atoms of this alkoxycarbonyl group is preferably in a range of 2 to 10, more preferably in a range of 2 to 6, still more preferably in a range of 2 to 4, and particularly preferably 2 or 3.

The non-fluorinated alkyl group which can be employed as A¹ or A² is an unsubstituted alkyl group or an alkyl group including a substituent other than a fluorine atom and may be linear or branched. Examples of the substituent other than a fluorine atom include those other than a fluorine atom from among substituents selected from the following substituent group Z. Among these, a hydroxy group, a carboxy group, a sulfo group, a sulfamoyl group (preferably an unsubstituted sulfamoyl group), or a carbamoyl group (preferably an unsubstituted carbamoyl group) is preferable.

The number of carbon atoms of the non-fluorinated alkyl group which can be employed as A¹ or A² is preferably in a range of 1 to 5 and more preferably in a range of 1 to 3. Further, methyl or ethyl is still more preferable and methyl is particularly preferable as this non-fluorinated alkyl group.

The number of carbon atoms of the aryl group which can be employed as A¹ or A² is preferably in a range of 6 to 20, more preferably in a range of 6 to 15, and still more preferably in a range of 6 to 12. Further, a phenyl group is even still more preferable as the aryl group. Such a phenyl group may include a substituent and a halogen atom (preferably a fluorine atom), a hydroxy group, a carboxy group, a sulfo group, or a sulfamoyl group (preferably an unsubstituted sulfamoyl group) is preferable as such a substituent.

In a case where the gas separation layer contains the polyimide compound having a structural portion represented by Formula (1), the gas permeability can be greatly improved while sufficiently improving the gas separation selectivity of the gas separation membrane to be obtained. Further, the plasticity resistance can be effectively improved. The reason for this is not clear, but a moderate degree of voids are generated between molecules and the affinity between the polyimide compound and CO₂ is also increased by introducing a CF₃ group which is bulky and rich in affinity for CO₂ into the polyimide compound, and a hydrogen bond is further effectively formed between polymers so that the polyimide compound is appropriately densified in the layer in a case where a hydroxy group is present on the same carbon as that in such a CF₃ group. As the result, the gas separation selectivity is improved and the layer becomes unlikely to be swollen even in a case of being brought into contact with impurity components such as toluene, and this is assumed to be one reason.

Further, in a case where the structural portion represented by Formula (1) contains a fluorinated alkyl group as A¹ or A², the gas separation selectivity tends to be degraded, and it becomes difficult to increase the plasticity resistance to a desired level. The reason for this is assumed to be an increase in hydrophobicity due to the coexistence of the fluorinated alkyl group as A¹ or A² and CF³ in Formula (1) on the same carbon and degradation of hydrogen bonding properties of the hydroxy group.

It is preferable that the polyimide compound used in the present invention has a unit structure represented by Formula (2).

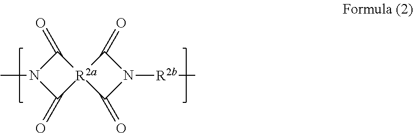

In Formula (2), R^{2a} represents a tetravalent linking group and R^{2b} represents a divalent linking group. Here, at least one of R^{2a} or R^{2b} has the structural portion represented by Formula (1).

In the unit structure represented by Formula (2), it is more preferable that both of R^{2a} and R^{2b} have the structural portion represented by Formula (1).

In a case where R^{2a} in Formula (2) has the structural portion represented by Formula (1), it is preferable that R^{2a} has a structure represented by any of Formulae (3-1) to (3-3).

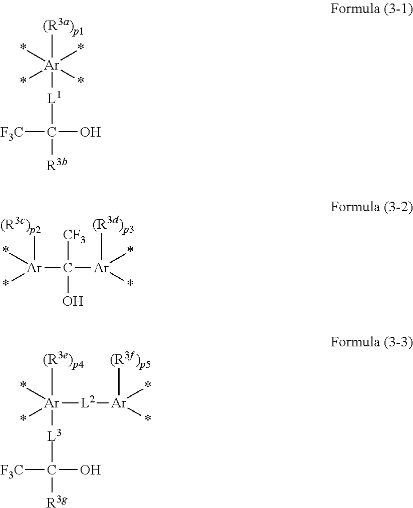

In Formulae (3-1) to (3-3), Ar represents an aromatic ring. The symbol “*” represents a linking site. L¹, L², and L³ represent a single bond or a divalent linking group. R^{3a}, R^{3c}, R^{3d}, R^{3e}, and R^{3f} represent a substituent. R^{3b} and R^{3g} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group. p1 to p5 represent an integer of 0 to 20.

As the divalent linking group which can be employed as L¹, L², and L³, —C(R^{x})₂— (R^{x} represents a hydrogen atom or a substituent, and in a case where R^{x} represents a substituent, R^{x}'s may be linked to each other to form a ring), —O—, —SO₂—, —C(═O)—, —S—, —NR^{Y}— (R^{Y} represents a hydrogen atom, an alkyl group (preferably a methyl group or an ethyl group), an aryl group (preferably a phenyl group)), —C₆H₄— (a phenylene group), or a combination of these is preferable.

L¹, L², and L³ respectively have a molecular weight of preferably 0 to 100 and more preferably 0 to 30.

L¹ and L³ represent preferably a single bond, an alkylene group, or an arylene group and more preferably a single bond.

It is more preferable that L² represents a single bond, —O—, or —C(R^{x})₂—. In a case where R^{x} represents a substituent, specific examples thereof include groups selected from the following substituent group Z. Among these, an alkyl group (the preferable range is the same as that of the alkyl group in the substituent group Z described below) is preferable, an alkyl group having a halogen atom as a substituent is more preferable, and trifluoromethyl is particularly preferable.

R^{3a}, R^{3c}, R^{3d}, R^{3e}, and R^{3f} represent a substituent, and R^{3c} and R^{3d}, and R^{3e} and R^{3f} may be linked to each other. Specific examples of such a substituent include groups selected from the following substituent group Z. Among these, an alkyl group or an aryl group is preferable and —CR^{X1}R^{X2}R^{X3} is more preferable. In a case where R^{X1}, R^{X2}, and R^{X3} represent a hydrogen atom or a substituent and adjacent two from among R^{X1}, R^{X2}, and R^{X3} represent a substituent, these may be linked to each other to form a ring. Further, it is preferable that one of R^{X1}, R^{X2}, and R^{X3} represents a halogenated alkyl group (preferably a fluorinated alkyl group and more preferably trifluoromethyl), another one thereof represents a hydroxy group, and the rest represents a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group. The preferable forms of the halogen atom, the carbamoyl group, the acyl group, the acyloxy group, the sulfamoyl group, the alkylsulfinyl group, the arylsulfinyl group, the alkylsulfonyloxy group, the alkoxycarbonyl group, the non-fluorinated alkyl group, and the aryl group are respectively the same as the preferable forms of the halogen atom, the carbamoyl group, the acyl group, the acyloxy group, the sulfamoyl group, the alkylsulfinyl group, the arylsulfinyl group, the alkylsulfonyloxy group, the alkoxycarbonyl group, the non-fluorinated alkyl group, and the aryl group as A¹ or A² described above.

The preferable forms of the halogen atom, the carbamoyl group, the acyl group, the acyloxy group, the sulfamoyl group, the alkylsulfinyl group, the arylsulfinyl group, the alkylsulfonyloxy group, the alkoxycarbonyl group, the non-fluorinated alkyl group, and the aryl group which can be employed as R^{3b} and R^{3g} are respectively the same as the preferable forms of the halogen atom, the carbamoyl group, the acyl group, the acyloxy group, the sulfamoyl group, the alkylsulfinyl group, the arylsulfinyl group, the alkylsulfonyloxy group, the alkoxycarbonyl group, the non-fluorinated alkyl group, and the aryl group which can be employed as A¹ or A² described above.

It is more preferable that R^{3b} and R^{3g} represent a hydrogen atom, a halogen atom, a carboxy group, a sulfo group, a sulfamoyl group, or a non-fluorinated alkyl group.

It is preferable that R^{3b} and R^{3g} represent a substituent having a low molecular weight with less steric hindrance or a group having hydrogen bonding properties. With such a form, a hydrogen bond can be further effectively formed between polyimide compounds and the plasticity resistance is further improved.

p1 to p5 represent preferably an integer of 0 to 10, more preferably 0 to 3, and still more preferably 0. The upper limit of p1 to p5 varies depending on the structure of Ar. In other words, the upper limit of p1 to p5 is the maximum value of the number of substituents which can be employed as Ar.

The aromatic ring Ar may be a single ring or a fused ring. Further, the aromatic ring Ar may be an aromatic hydrocarbon ring or an aromatic heterocyclic ring. Specific examples of the aromatic ring Ar include a benzene ring, a naphthalene ring, an anthracene ring, a fluorene ring, an indene ring, an indane ring, a triptycene ring, a xanthene ring, a furan ring, a thiophene ring, a pyrrole ring, a pyrazole ring, an imidazole ring, a pyridine ring, and a pyrimidine ring. Among these, a benzene ring is preferable.

In a case where Ar represents a benzene ring, p1 represents 0 or 1 and preferably 0. Further, p2, p3, and p5 represent an integer of 0 to 3, preferably 0 or 1, and more preferably 0. Further, p4 represents an integer of 0 to 2, preferably 0 or 1, and more preferably 0.

In a case where R^{2a} has the structural portion represented by Formula (1), the unit structure represented by Formula (2) is represented by any of Formulae (8) to (10).

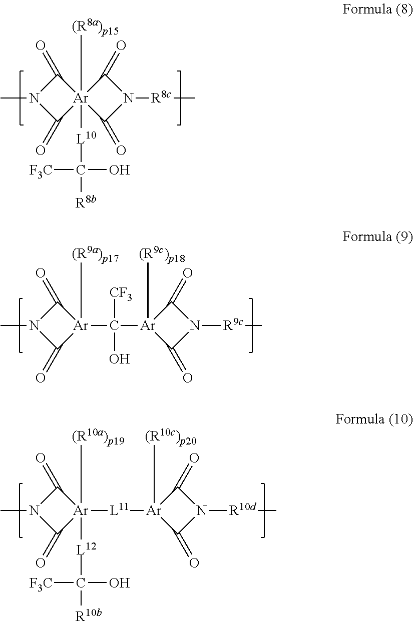

In Formulae (8) to (10), Ar represents an aromatic ring. R^{8a}, R^{9a}, R^{9b}, R^{10a}, and R^{10c} represent a substituent. R^{8b} and R^{10b} represent a hydrogen atom, a halogen atom, a carboxy group, a carbamoyl group, an acyl group, a carbonyloxy group, a sulfo group, a sulfamoyl group, a sulfinyl group, a sulfonyloxy group, a non-fluorinated alkyl group, or an aryl group. R^{8c}, R^{9c}, and R^{10d} have the same definition as that for R^{2b} in Formula (2) and the preferable forms are the same as described above. L¹⁰, L¹¹, and L¹² represent a single bond or a divalent linking group. p16 to p20 represent an integer of 0 to 20.

The preferable forms of R^{8a}, R^{9a}, R^{9b}, R^{10a}, and R^{10c} are respectively the same as the preferable forms of R^{3a}, R^{3c}, R^{3d}, R^{3e}, and R^{3f} in Formulae (3-1) to (3-3). The preferable forms of R^{8b} and R^{10b} are respectively the same as the preferable forms of R^{3b} and R^{3g} in Formulae (3-1) to (3-3). The preferable forms of L¹⁰, L¹¹, and L¹² are respectively the same as the preferable forms of L¹, L², and L³ in Formulae (3-1) to (3-3). The preferable forms of p16 to p20 are respectively the same as the preferable forms of p1 to p5 in formulae (3-1) to (3-3).

The preferable form of Ar is the same as the preferable form of Ar in Formulae (3-1) to (3-3).

It is preferable that the polyimide compound of the present invention includes at least a substituent having a structure represented by Formula (1) on Ar.

In a case where R^{2b} in Formula (2) has the structural portion represented by Formula (1), it is preferable that the structure of R^{2b} is a structure represented by any of Formulae (4-1) to (4-3).

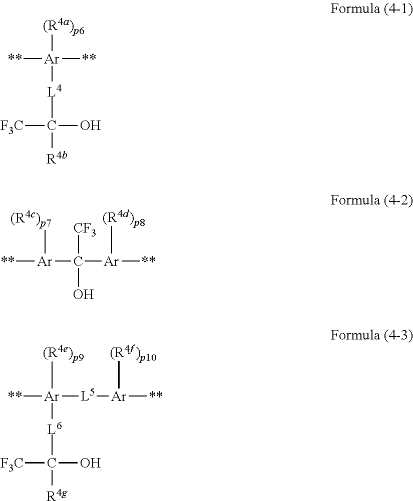

In Formulae (4-1) to (4-3), Ar represents an aromatic ring. The symbol “**” represents a linking site. L⁴, L⁵, and L⁶ represent a single bond or a divalent linking group. R^{4a}, R^{4c}, R^{4d}, R^{4e}, and R^{4f} represent a substituent. R^{4b} and R^{4g} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group. p6 to p10 represent an integer of 0 to 20.

L⁴, L⁵, and L⁶ each have the same definition as that for L¹, L², and L³ in Formulae (3-1) to (3-3), and the preferable forms thereof are the same as described above. R^{4b} and R^{4g} each have the same definition as that for R^{3b} and R^{3g} in Formulae (3-1) to (3-3), and the preferable forms are the same as described above.

The preferable form of Ar is the same as the preferable form of Ar in Formulae (3-1) to (3-3).

In a case where Ar represents a benzene ring, p6 and p9 represent an integer of 0 to 3, preferably 0 to 2, and more preferably 0 or 1. Further, p7, p8, and p10 represent an integer f 0 to 4, more preferably 0 to 3, still more preferably 0 to 2, and particularly preferably 0 or 1.

R^{4a}, R^{4c}, R^{4d}, R^{4e}, and R^{4f} represent a substituent and specific examples thereof include groups selected from the following substituent group Z. Among these, an alkyl group (preferably an alkyl group having 1 to 5 carbon atoms and more preferably methyl or ethyl) is preferable.

p6 to p10 represent preferably 0 to 10, more preferably 0 to 3, and still more preferably 0 or 1.

In a case where R^{2b} has a structure represented by Formula (1), it is preferable that the unit structure represented by Formula (2) is represented by any of Formulae (5) to (7).

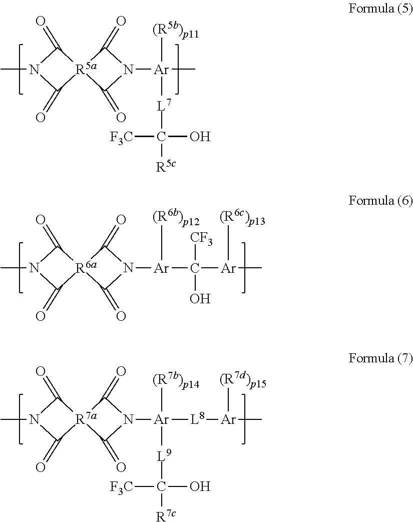

In Formulae (5) to (7), Ar represents an aromatic ring. R^{5a}, R^{6a}, and R^{7a} have the same definition as that for R^{2a} in Formula (2) and the preferable forms thereof are the same as described above. R^{5b}, R^{6b}, R^{6c}, R^{7b}, and R^{7d} represent a substituent. R^{5c} and R^{7c} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group. L⁷, L⁸, and L⁹ represent a single bond or a divalent linking group. p11 to p15 represent an integer of 0 to 20.

The preferable forms of R^{5b}, R^{6b}, R^{6c}, R^{7b}, and R^{7d} are respectively the same as the preferable forms of R^{4a}, R^{4c}, R^{4d}, R^{4e}, and R^{4f} in Formulae (4-1) to (4-3). The preferable forms of R^{5c} and R^{7c} are respectively the same as the preferable forms of R^{4b} and R^{4g} in Formulae (4-1) to (4-3). The preferable forms of L⁷, L⁸, and L⁹ are respectively the same as the preferable forms of L⁴, L⁵, and L⁶ in Formulae (4-1) to (4-3). The preferable forms of p11 to p15 are respectively the same as the preferable forms of p6 to p10 in formulae (4-1) to (4-3).

The preferable form of Ar is the same as the preferable form of Ar in Formulae (4-1) to (4-3).

It is preferable that the polyimide compound of the present invention includes at least a substituent having a structure represented by Formula (1) on Ar.

In the polyimide compound used in the present invention, in a case where R^{2a} in Formula (2) does not have the structural portion represented by Formula (1), it is preferable that the structure of R^{2a} is represented by any of Formulae (I-1) to (I-28).

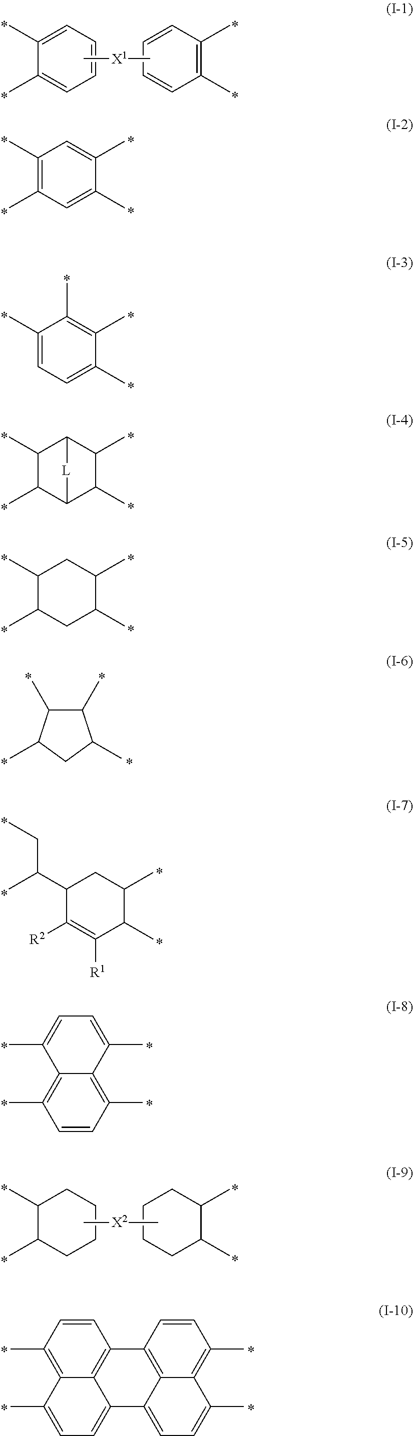

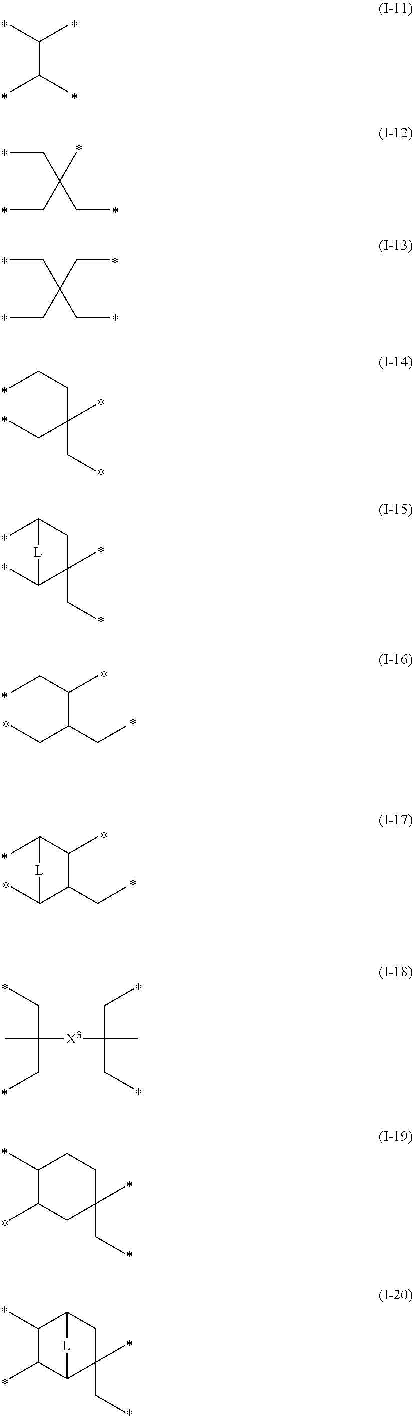

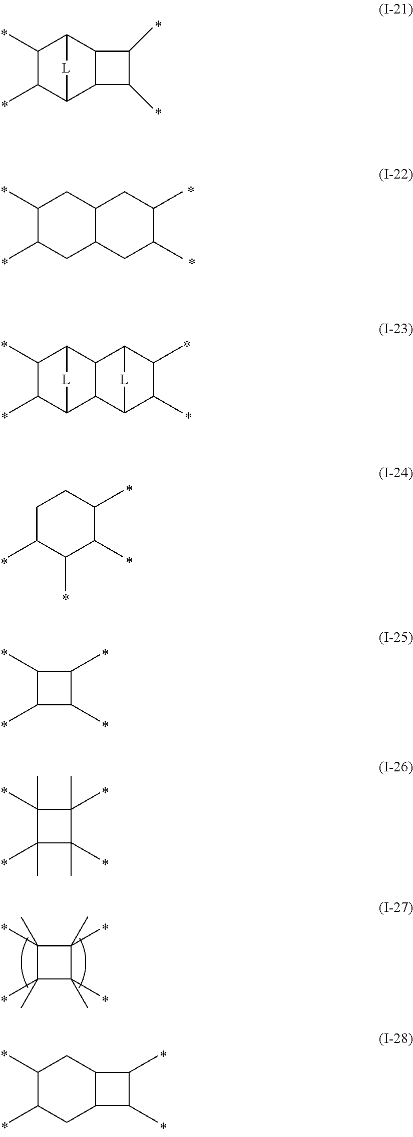

In Formulae (I-1) to (I-28), X¹ to X³ represent a single bond or a divalent linking group. L represents —CH═CH— or —CH₂—. R¹ and R² represent a hydrogen atom or a substituent that does not have a structural portion represented by Formula (1). The symbol “*” represents a linking site.

As the divalent linking group, —C(R^{x})₂— (R^{x} represents a hydrogen atom or a substituent, and in a case where R^{x} represents a substituent, R's may be linked to each other to form a ring), —O—, —SO₂—, —C(═O)—, —S—, —NR^{Y}— (R^{Y} represents a hydrogen atom, an alkyl group (preferably a methyl group or an ethyl group), an aryl group (preferably a phenyl group)), —C₆H₄— (a phenylene group), or a combination of these is preferable. It is more preferable that X¹ to X³ represent a single bond or —C(R^{x})₂—. In a case where R^{x} represents a substituent, specific examples thereof include groups selected from the following substituent group Z. Among these, an alkyl group (the preferable range is the same as that of the alkyl group in the substituent group Z described below) is preferable, an alkyl group having a halogen atom as a substituent is more preferable, and trifluoromethyl is particularly preferable. Moreover, in Formula (I-18), X³ is linked to any one of two carbon atoms shown on the left side thereof and any one of two carbon atoms shown on the right side thereof.

In Formulae (I-4), (I-15), (I-17), (I-20), (I-21), and (I-23), L represents —CH═CH— or —CH₂—.

R¹ and R² represent a hydrogen atom or a substituent that does not have the structural portion represented by Formula (1). Examples of such a substituent include groups selected from the substituent group Z described below. R¹ and R² may be bonded to each other to form a ring.

R¹ and R² represent preferably a hydrogen atom or a non-fluorinated alkyl group, more preferably a hydrogen atom, a methyl group, or an ethyl group, and still more preferably a hydrogen atom.

The carbon atoms shown in Formulae (I-1) to (I-28) may further include a substituent. Specific examples of the substituent include groups selected from the substituent group Z described below. Among these, an alkyl group or an aryl group is preferable.

In the polyimide compound used in the present invention, in a case where R^{2b} in Formula (2) does not have the structural portion represented by Formula (1), it is preferable that the structure of R^{2b} is represented by Formula (II-a) or (II-b).

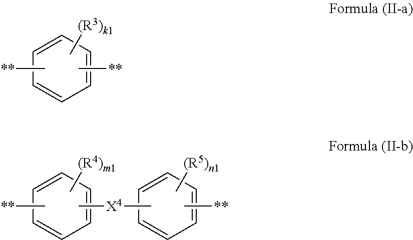

R³, R⁴, and R⁵ represent a substituent that does not have the structural portion represented by Formula (1). Specific examples of the substituent include groups selected from groups that do not have a structure represented by Formula (1) from among the following substituent group Z. Among these, a non-fluorinated alkyl group or a carboxy group is preferable, and a methyl group, an ethyl group, or a carboxy group is more preferable.

R⁴ and R⁵ may be linked to each other to form a ring together with X⁴. The structure formed by R⁴ and R⁵ being linked to each other is not particularly limited, but a single bond, —O—, or —S— is preferable.

k1 represents an integer of 0 to 4, preferably 1 to 4, and more preferably 4.

m1 and n1 represent an integer of 0 to 4, preferably 1 to 4, and more preferably 4.

X⁴ has the same definition as that for X¹ to X³ in Formulae (I-1) to (I-28) and the preferable forms thereof are the same as described above.

The polyimide compound used in the present invention may have a unit structure represented by Formula (2a), which does not have the structural portion represented by Formula (1), in addition to the unit structure represented by Formula (2). The polyimide compound may have a unit structure represented by Formula (2) which has the structural portion represented by Formula (1) and a unit structure represented by Formula (2) which does not have the structural portion represented by Formula (1).

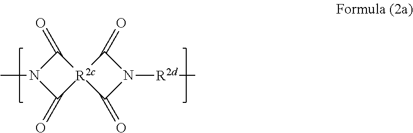

In Formula (2a), R^{2C} represents a structure represented by any of Formulae (I-1) to (I-28), and the preferable forms are the same as the preferable forms in a case where R^{2a} in Formula (2) does not have the structural portion represented by Formula (1). R^{2d} represents a structure represented by any of Formulae (II-a) or (II-b8), and the preferable forms are the same as the preferable forms in a case where R^{2b} in Formula (2) does not have the structure represented by Formula (1).

In the structure of the polyimide compound used in the present invention, the ratio of the molar amount of the repeating unit represented by Formula (2) to the total molar amount of the repeating unit represented by Formula (2) and the repeating unit represented by Formula (2a) is preferably in a range of 50% to 100% by mole, more preferably in a range of 70% to 100% by mole, still more preferably in a range of 80% to 100% by mole, and even still more preferably in a range of 90% to 100% by mole. Further, the expression “the ratio of the molar amount of the repeating unit represented by Formula (2) to the total molar amount of the repeating unit represented by Formula (2) and the repeating unit represented by Formula (2a) is 100% by mole” means that the polyimide compound does not have the repeating unit represented by Formula (2a).

It is preferable that the polyimide compound used in the present invention contains 0.50 mol of the structural portion represented by Formula (1) in 1 g of the polyimide compound in terms of dry mass (in other words, it is preferable that the content of the structural portion represented by Formula (1) in the polyimide compound is preferably 0.50 mol/g or greater).

The dry mass of the polyimide compound indicates the mass of the polyimide compound synthesized according to the scheme described below in the example after being dried at 40° C. and a relative humidity of 20% for 18 hours.

The content of the structural portion represented by Formula (1) in the polyimide compound used in the present invention is more preferably 1.00 mol/g or greater and still more preferably 1.50 mol/g or greater. The upper limit of the content of the structural portion represented by Formula (1) in the polyimide compound used in the present invention is not particularly limited, and is practically 30.00 mol/g or less and typically 10.00 mol/g or less.

Examples of the substituent group Z include:

an alkyl group (the number of carbon atoms of the alkyl group is preferably in a range of 1 to 30, more preferably in a range of 1 to 20, and particularly preferably in a range of 1 to 10, and examples thereof include methyl, ethyl, iso-propyl, tert-butyl, n-octyl, n-decyl, and n-hexadecyl), a cycloalkyl group (the number of carbon atoms of the cycloalkyl group is preferably in a range of 3 to 30, more preferably in a range of 3 to 20, and particularly preferably in a range of 3 to 10, and examples thereof include cyclopropyl, cyclopentyl, and cyclohexyl), an alkenyl group (the number of carbon atoms of the alkenyl group is preferably in a range of 2 to 30, more preferably in a range of 2 to 20, and particularly preferably in a range of 2 to 10, and examples thereof include vinyl, allyl, 2-butenyl, and 3-pentenyl), an alkynyl group (the number of carbon atoms of the alkynyl group is preferably in a range of 2 to 30, more preferably in a range of 2 to 20, and particularly preferably in a range of 2 to 10, and examples thereof include propargyl and 3-pentynyl), an aryl group (the number of carbon atoms of the aryl group is preferably in a range of 6 to 30, more preferably in a range of 6 to 20, and particularly preferably in a range of 6 to 12, and examples thereof include phenyl, p-methylphenyl, naphthyl, and anthranyl), an amino group (such as an amino group, an alkylamino group, an arylamino group, or a heterocyclic amino group; the number of carbon atoms of the amino group is preferably in a range of 0 to 30, more preferably in a range of 0 to 20, and particularly preferably in a range of 0 to 10 and examples thereof include amino, methylamino, dimethylamino, diethylamino, dibenzylamino, diphenylamino, and ditolylamino), an alkoxy group (the number of carbon atoms of the alkoxy group is preferably in a range of 1 to 30, more preferably in a range of 1 to 20, and particularly preferably in a range of 1 to 10, and examples thereof include methoxy, ethoxy, butoxy, and 2-ethylhexyloxy), an aryloxy group (the number of carbon atoms of the aryloxy group is preferably in a range of 6 to 30, more preferably in a range of 6 to 20, and particularly preferably in a range of 6 to 12, and examples thereof include phenyloxy, 1-naphthyloxy, and 2-naphthyloxy), a heterocyclic oxy group (the number of carbon atoms of the heterocyclic oxy group is preferably in a range of 1 to 30, more preferably in a range of 1 to 20, and particularly preferably in a range of 1 to 12, and examples thereof include pyridyloxy, pyrazyloxy, pyrimidyloxy, and quinolyloxy),

an acyl group (the number of carbon atoms of the acyl group is preferably in a range of 1 to 30, more preferably in a range of 1 to 20, and particularly preferably in a range of 1 to 12, and examples thereof include acetyl, benzoyl, formyl, and pivaloyl), an alkoxycarbonyl group (the number of carbon atoms of the alkoxycarbonyl group is preferably in a range of 2 to 30, more preferably in a range of 2 to 20, and particularly preferably in a range of 2 to 12, and examples thereof include methoxycarbonyl and ethoxycarbonyl), an aryloxycarbonyl group (the number of carbon atoms of the aryloxycarbonyl group is preferably in a range of 7 to 30, more preferably in a range of 7 to 20, and particularly preferably in a range of 7 to 12, and examples thereof include phenyloxycarbonyl), an acyloxy group (the number of carbon atoms of the acyloxy group is preferably in a range of 2 to 30, more preferably in a range of 2 to 20, and particularly preferably in a range of 2 to 10, and examples thereof include acetoxy and benzoyloxy), an acylamino group (the number of carbon atoms of the acylamino group is preferably in a range of 2 to 30, more preferably in a range of 2 to 20, and particularly preferably in a range of 2 to 10, and examples thereof include acetylamino and benzoylamino),

an alkoxycarbonylamino group (the number of carbon atoms of the alkoxycarbonylamino group is preferably in a range of 2 to 30, more preferably in a range of 2 to 20, and particularly preferably in a range of 2 to 12, and examples thereof include methoxycarbonylamino), an aryloxycarbonylamino group (the number of carbon atoms of the aryloxycarbonylamino group is preferably in a range of 7 to 30, more preferably in a range of 7 to 20, and particularly preferably in a range of 7 to 12, and examples thereof include phenyloxycarbonylamino), a sulfonylamino group (the number of carbon atoms of the sulfonylamino group is preferably in a range of 1 to 30, more preferably in a range of 1 to 20, and particularly preferably in a range of 1 to 12, and examples thereof include methanesulfonylamino and benzenesulfonylamino), a sulfamoyl group (the number of carbon atoms of the sulfamoyl group is preferably in a range of 0 to 30, more preferably in a range of 0 to 20, and particularly preferably in a range of 0 to 12, and examples thereof include sulfamoyl, methylsulfamoyl, dimethylsulfamoyl, and phenylsulfamoyl),

an alkylthio group (the number of carbon atoms of the alkylthio group is preferably in a range of 1 to 30, more preferably in a range of 1 to 20, and particularly preferably in a range of 1 to 12, and examples thereof include methylthio and ethylthio), an arylthio group (the number of carbon atoms of the arylthio group is preferably in a range of 6 to 30, more preferably in a range of 6 to 20, and particularly preferably in a range of 6 to 12, and examples thereof include phenylthio), a heterocyclic thio group (the number of carbon atoms of the heterocyclic thio group is preferably in a range of 1 to 30, more preferably in a range of 1 to 20, and particularly preferably in a range of 1 to 12, and examples thereof include pyridylthio, 2-benzimidazolylthio, 2-benzoxazolylthio, and 2-benzothiazolylthio),

a sulfonyl group (the number of carbon atoms of the sulfonyl group is preferably in a range of 1 to 30, more preferably in a range of 1 to 20, and particularly preferably in a range of 1 to 12, and examples thereof include mesyl and tosyl), a sulfinyl group (the number of carbon atoms of the sulfinyl group is preferably in a range of 1 to 30, more preferably in a range of 1 to 20, and particularly preferably in a range of 1 to 12, and examples thereof include methanesulfinyl and benzenesulfinyl), an ureido group (the number of carbon atoms of the ureido group is preferably in a range of 1 to 30, more preferably in a range of 1 to 20, and particularly preferably in a range of 1 to 12, and examples thereof include ureido, methylureido, and phenylureido), a phosphoric acid amide group (the number of carbon atoms of the phosphoric acid amide group is preferably in a range of 1 to 30, more preferably in a range of 1 to 20, and particularly preferably in a range of 1 to 12, and examples thereof include diethyl phosphoric acid amide and phenyl phosphoric acid amide), a hydroxy group, a mercapto group, a halogen atom (such as a fluorine atom, a chlorine atom, a bromine atom, or an iodine atom, and a fluorine atom is more preferable),

a cyano group, a carboxy group, an oxo group, a nitro group, a hydroxamic acid group, a sulfino group, a hydrazine group, an imino group, a heterocyclic group (a 3- to 7-membered ring heterocyclic group is preferable, the hetero ring may be aromatic or non-aromatic, examples of a heteroatom constituting the hetero ring include a nitrogen atom, an oxygen atom, and a sulfur atom, the number of carbon atoms of the heterocyclic group is preferably in a range of 0 to 30 and more preferably in a range of 1 to 12, and specific examples thereof include imidazolyl, pyridyl, quinolyl, furyl, thienyl, piperidyl, morpholino, benzoxazolyl, benzimidazolyl, benzothiazolyl, carbazolyl, and azepinyl), a silyl group (the number of carbon atoms of the silyl group is preferably in a range of 3 to 40, more preferably in a range of 3 to 30, and particularly preferably in a range of 3 to 24, and examples thereof include trimethylsilyl and triphenylsilyl), and a silyloxy group (the number of carbon atoms of the silyloxy group is preferably in a range of 3 to 40, more preferably in a range of 3 to 30, and particularly preferably in a range of 3 to 24, and examples thereof include trimethylsilyloxy and triphenylsilyloxy). These substituents may be substituted with any one or more substituents selected from the substituent group Z.

Further, in the present invention, in a case where a plurality of substituents are present at one structural site, these substituents may be linked to each other to form a ring or may be condensed with some or entirety of the structural site and form an aromatic ring or an unsaturated hetero ring.

In a case where a compound or a substituent includes an alkyl group or an alkenyl group, these may be linear or branched and may be substituted or unsubstituted. In addition, in a case where a compound or a substituent includes an aryl group or a heterocyclic group, these may be a single ring or a condensed ring and may be substituted or unsubstituted.

In the present specification, in a case where a group is described as only a substituent, the substituent group Z can be used as reference unless otherwise specified. Further, in a case where only the names of the respective groups are described (for example, a group is described as an “alkyl group”), the preferable range and the specific examples of the corresponding group in the substituent group Z are applied.

The molecular weight of the polyimide compound used in the present invention is preferably in a range of 10,000 to 1,000,000, more preferably in a range of 15,000 to 500,000, and still more preferably in a range of 20,000 to 200,000 as the weight-average molecular weight.

The molecular weight and the dispersity in the present specification are set to values measured using a gel permeation chromatography (GPC) method unless otherwise specified and the molecular weight is set to a weight-average molecular weight in terms of polystyrene. A gel including an aromatic compound as a repeating unit is preferable as a gel filling a column used for the GPC method and examples of the gel include a gel formed of a styrene-divinylbenzene copolymer. It is preferable that two to six columns are linked to each other and used. Examples of a solvent to be used include an ether-based solvent such as tetrahydrofuran and an amide-based solvent such as N-methylpyrrolidinone. It is preferable that measurement is performed at a flow rate of the solvent of 0.1 to 2 mL/min and most preferable that the measurement is performed at a flow rate thereof of 0.3 to 1.5 mL/min. In a case where the measurement is performed in the above-described range, a load is not applied to the apparatus and the measurement can be more efficiently performed. The measurement temperature is preferably in a range of 10° C. to 50° C. and most preferably in a range of 20° C. to 40° C. In addition, the column and the carrier to be used can be appropriately selected according to the physical properties of a polymer compound which is a target for measurement.

(Synthesis of Polyimide Compound)

The polyimide compound used in the present invention can be synthesized by performing condensation and polymerization of a specific bifunctional acid anhydride (tetracarboxylic dianhydride) and a specific diamine. Such methods can be performed by referring to the technique described in a general book (for example, “The Latest Polyimide ˜Fundamentals and Applications˜” edited by Toshio Imai and Rikio Yokota, NTS Inc., Aug. 25, 2010, pp. 3 to 49) as appropriate.

At least one tetracarboxylic dianhydride serving as a raw material in synthesis of the polyimide compound used in the present invention is represented by Formula (IV). It is preferable that all tetracarboxylic dianhydrides which are the raw materials are represented by Formula (IV).

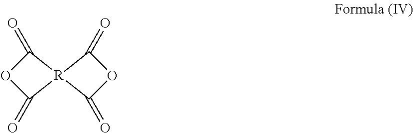

In Formula (IV), R has the same definition as that for R^{2a} in Formula (2).

In a case where Formula (IV) shows a structure represented by Formula (1), it is preferable that the structure is represented by any of Formulae (IV-1) to (IV-3).

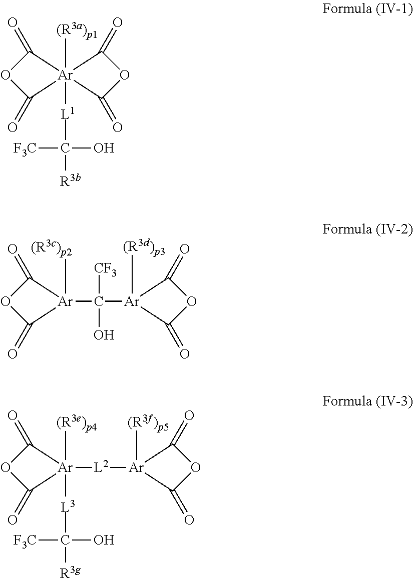

In Formulae (IV-1) to (IV-3), Ar, L1, L², L³, R^{3a}, R^{3b}, R^{3c}, R^{3d}, R^{3e}, R^{3f}, R^{3g}, and p1 to p5 each have the same definition as that for Ar, L1, L², L³, R^{3a}, R^{3b}, R^{3c}, R^{3d}, R^{3e}, R^{3f}, R^{3g}, and p1 to p5 in Formulae (3-1) to (3-3).

Specific examples of the tetracarboxylic dianhydride represented by any of Formulae (IV-1) to (IV-3) include compounds shown below.

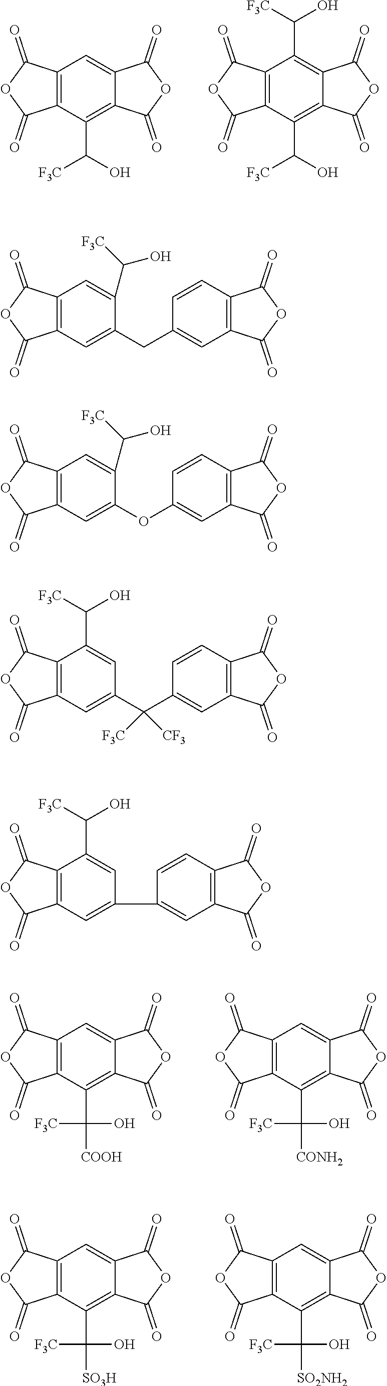

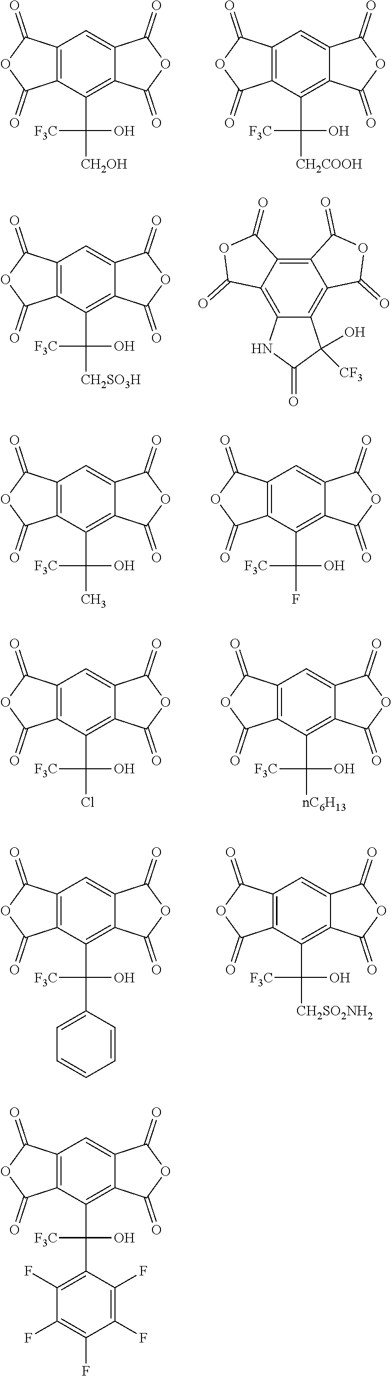

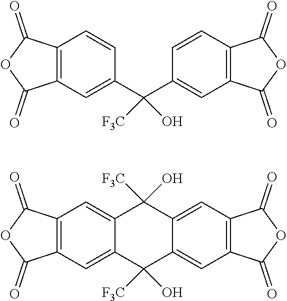 [SMILES: O=C1OC(=O)c2cc(C(O)(c3ccc4c(c3)C(=O)OC4=O)C(F)(F)F)ccc21 ; O=C1OC(=O)c2cc3c(cc21)C(O)(C(F)(F)F)c1cc2c(cc1C3(O)C(F)(F)F)C(=O)OC2=O]

In a case where Formula (IV) does not show the structural portion represented by Formula (1), specific examples of the tetracarboxylic dianhydride which can be used in the present invention include compounds shown below.

The polyimide compound can be synthesized using a tetracarboxylic dianhydride represented by Formula (IV) which has the structural portion represented by Formula (1) and a tetracarboxylic dianhydride represented by formula (IV) which does not have the structural portion represented by Formula (1).

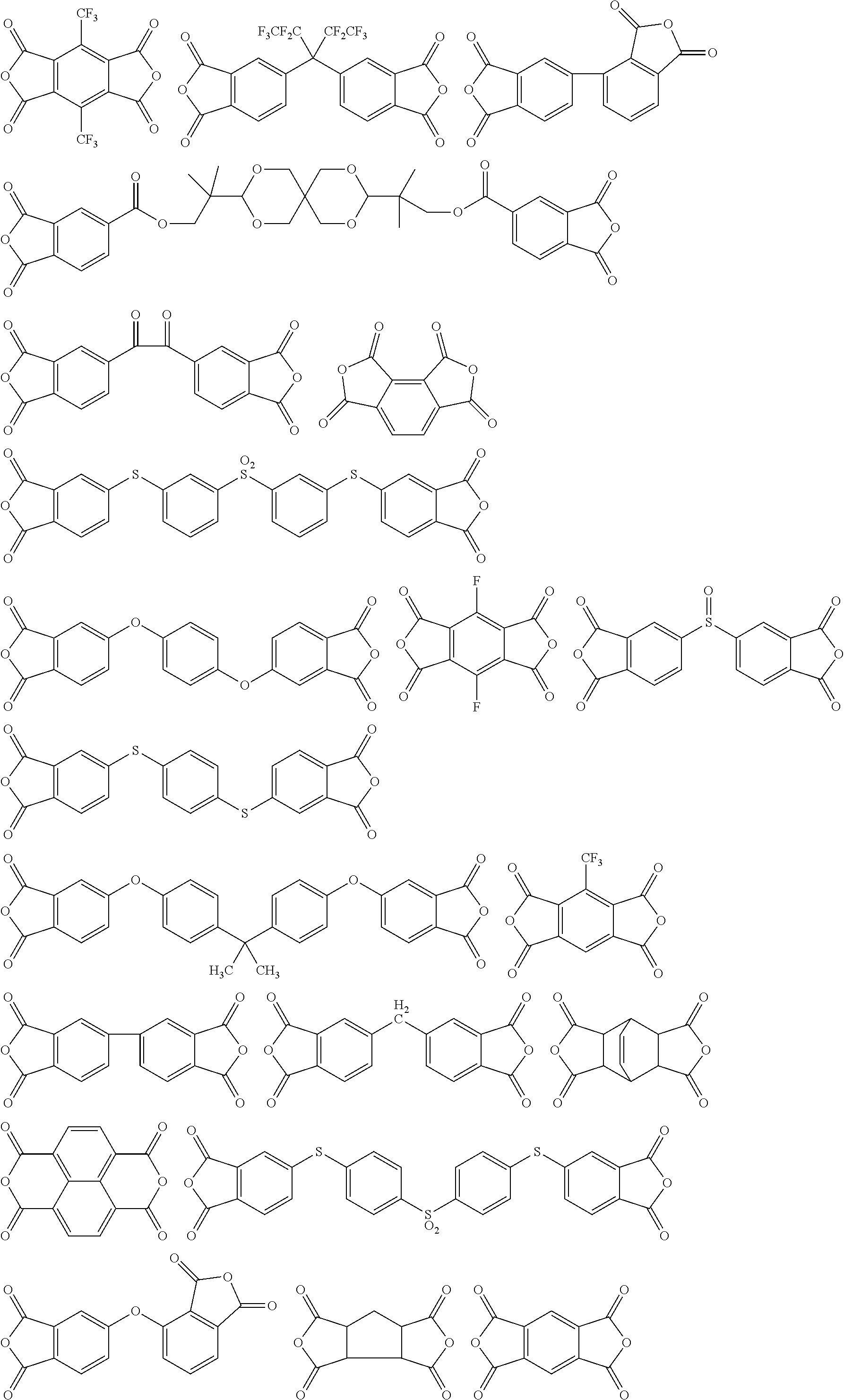

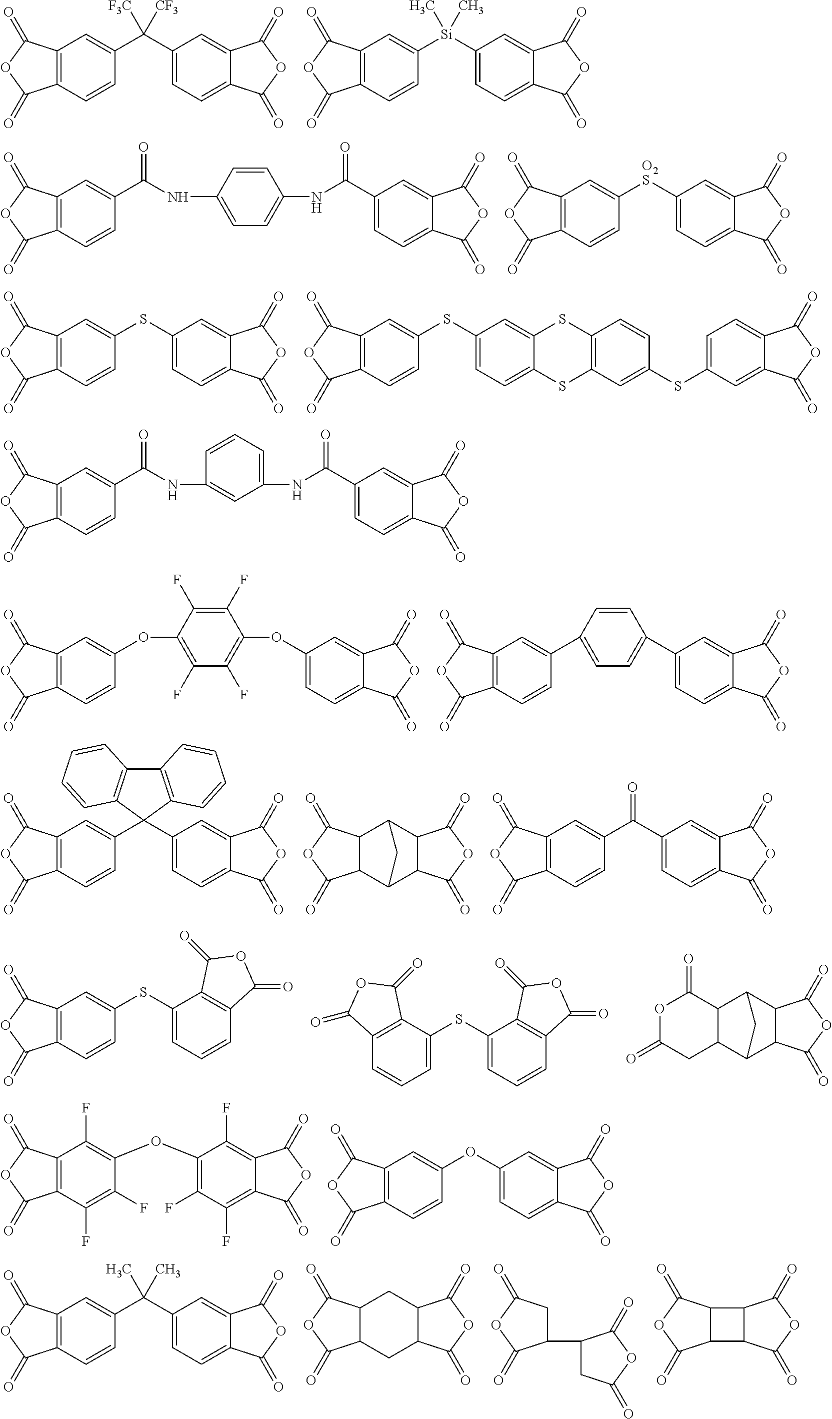

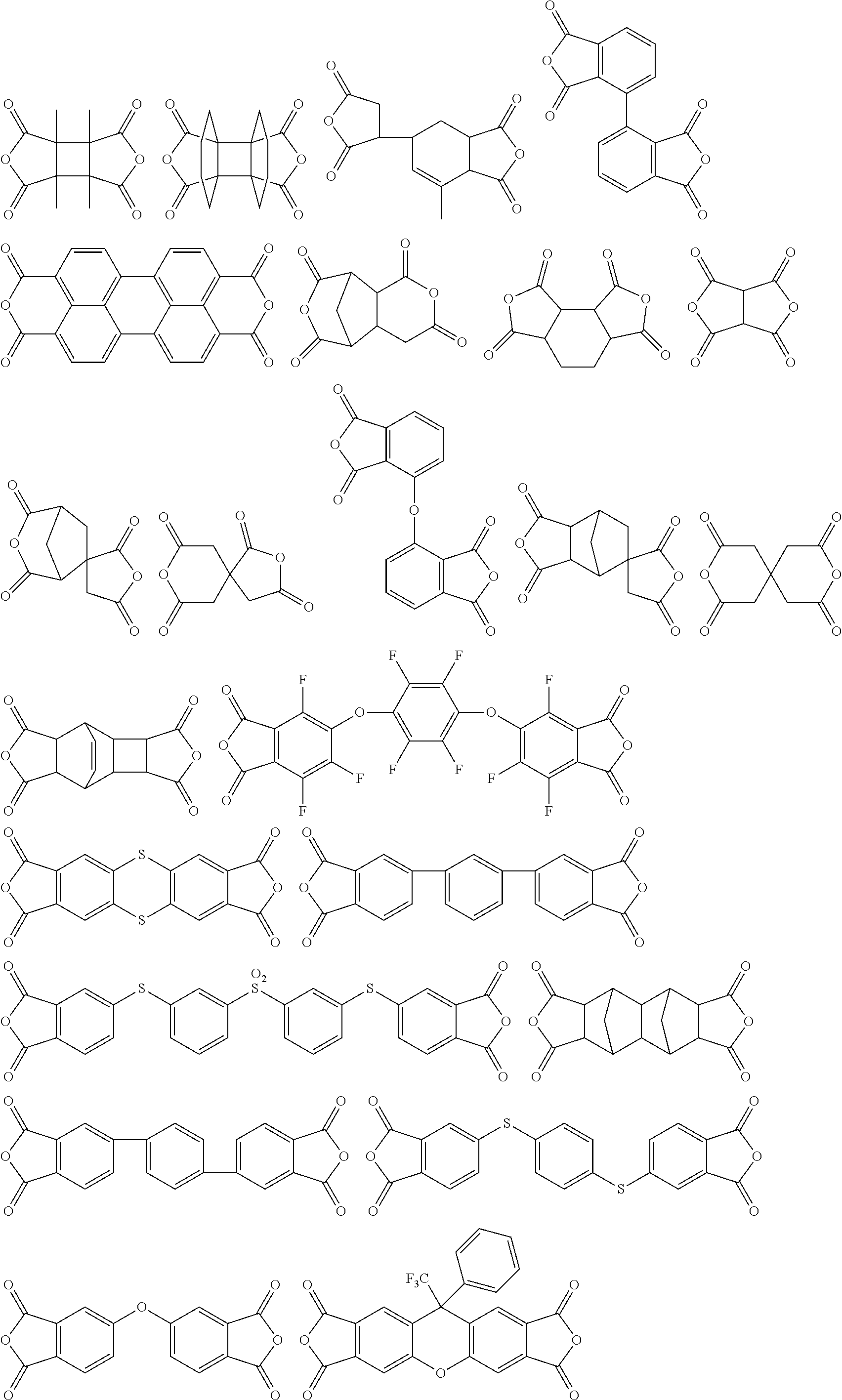

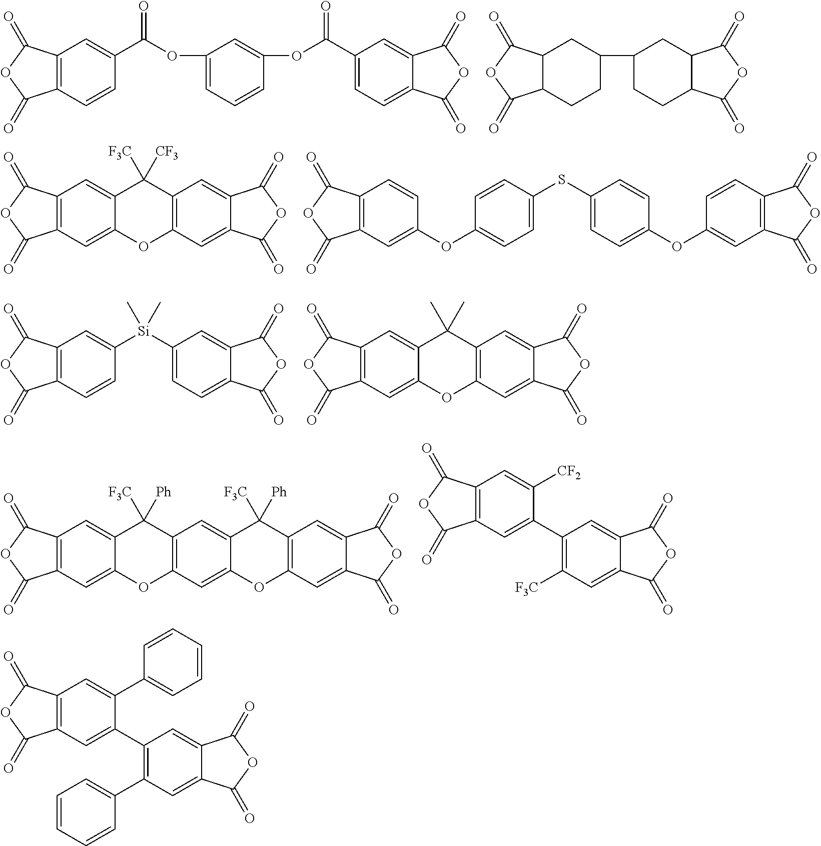

In the synthesis of the polyimide compound which can be used in the present invention, it is preferable that the diamine compound as another raw material is represented by any of Formulae (V-1) to (V-3) in a case where the diamine compound has the structural portion represented by Formula (1).

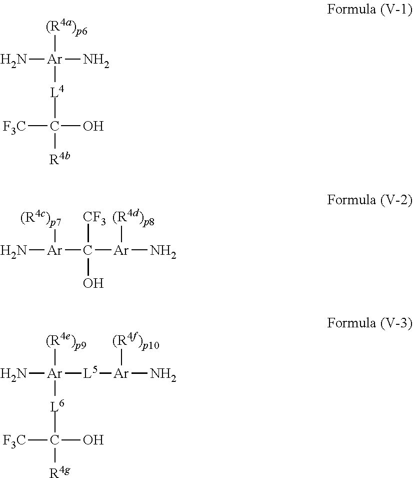

In Formulae (V-1) to (V-3), Ar, L⁴, L⁵, L⁶, R^{4a}, R^{4b}, R^{4c}, R^{4d}, R^{4e}, R^{4f}, R^{4g}, and p6 to p10 each have the same definition as that for Ar, L⁴, L⁵, L⁶, R^{4a}, R^{4b}, R^{4c}, R^{4d}, R^{4e}, R^{4f}, R^{4g}, and p6 to p10 in Formulae (4-1) to (4-3).

Specific examples of the diamine compound represented by Formulae (V-1) to (V-3) include compounds shown below, but the present invention is not limited to these.

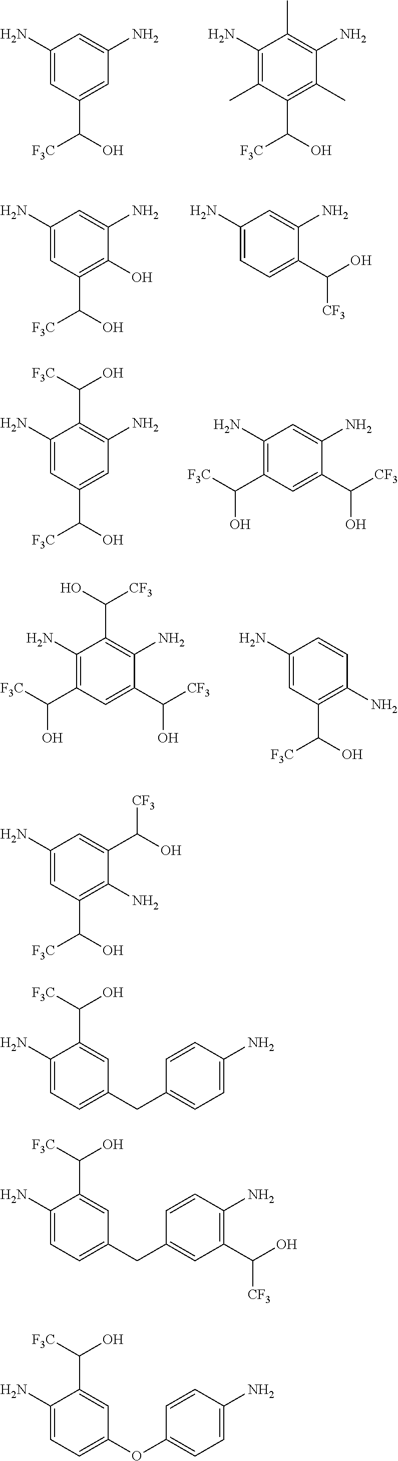

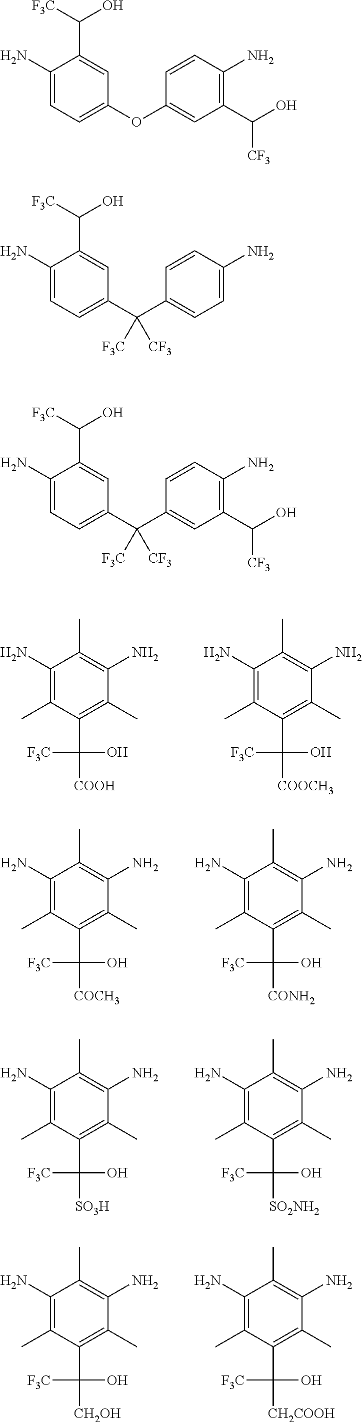

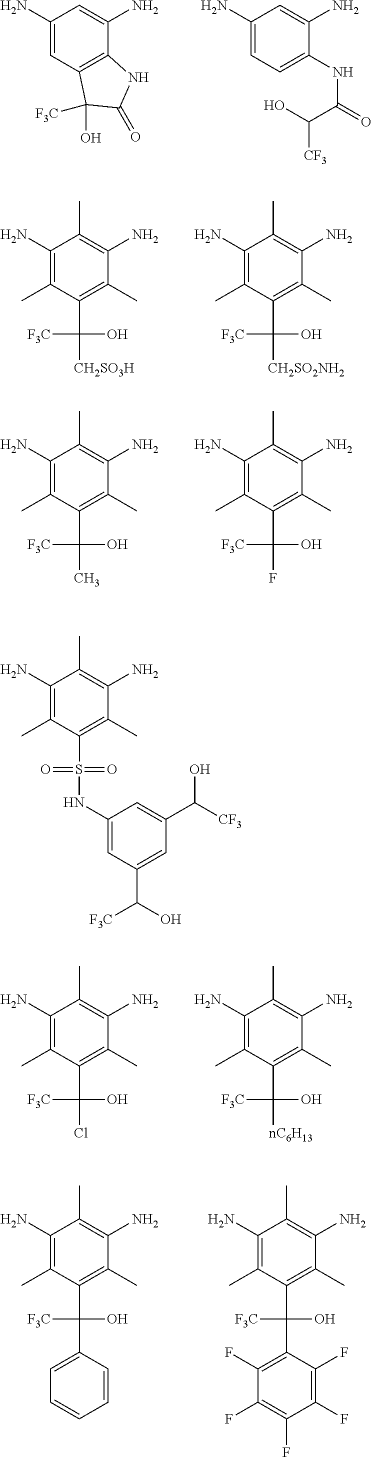

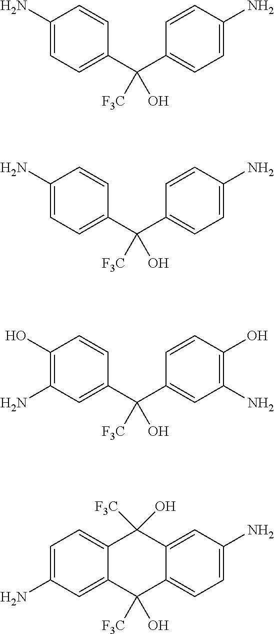 [SMILES: Nc1ccc(C(O)(c2ccc(N)cc2)C(F)(F)F)cc1 ; Nc1ccc(C(O)(c2ccc(N)cc2)C(F)(F)F)cc1 ; Nc1cc(C(O)(c2ccc(O)c(N)c2)C(F)(F)F)ccc1O ; Nc1ccc2c(c1)C(O)(C(F)(F)F)c1ccc(N)cc1C2(O)C(F)(F)F]

In the synthesis of the polyimide compound which can be used in the present invention, it is preferable that the diamine compound as a raw material is represented by Formula (VII-a) or (VII-b) in a case where R^{2b} in Formula (2) does not have the structural portion represented by Formula (1).

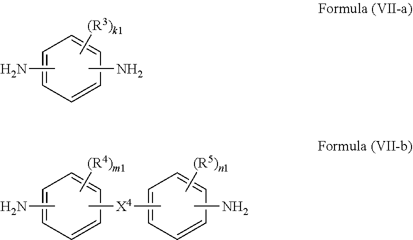

In Formula (VII-a), R³ and k1 each have the same definition as that for R³ and k1 in Formula (II-a).

In Formula (VII-b), R⁴, R⁵, X⁴, m1, and n1 each have the same definition as that for R⁴, R⁵, X⁴, m1, and n1 in Formula (II-b).

As the diamine compound represented by Formula (VII-a) or (VII-b), for example, the following compounds can be used.

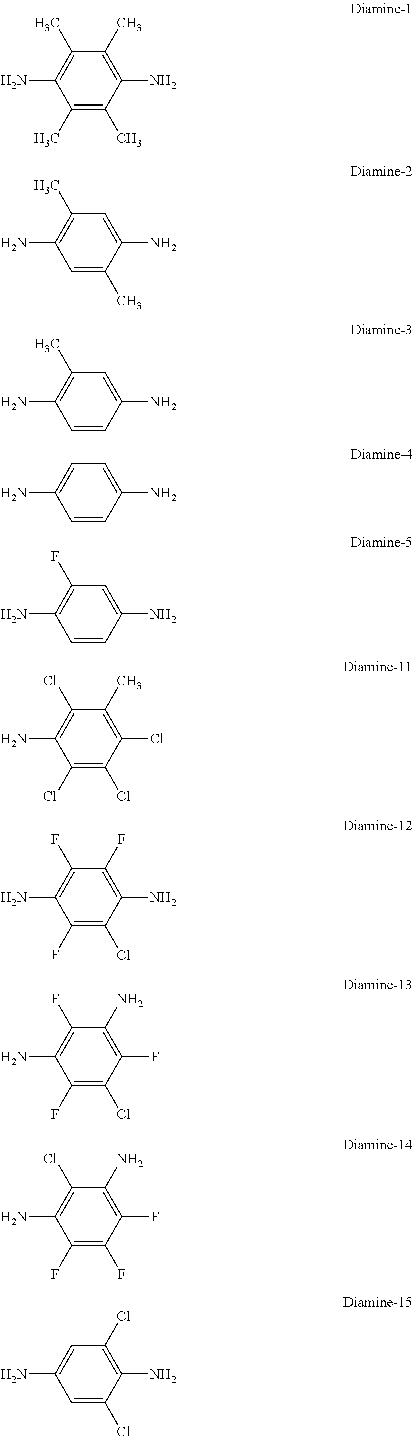

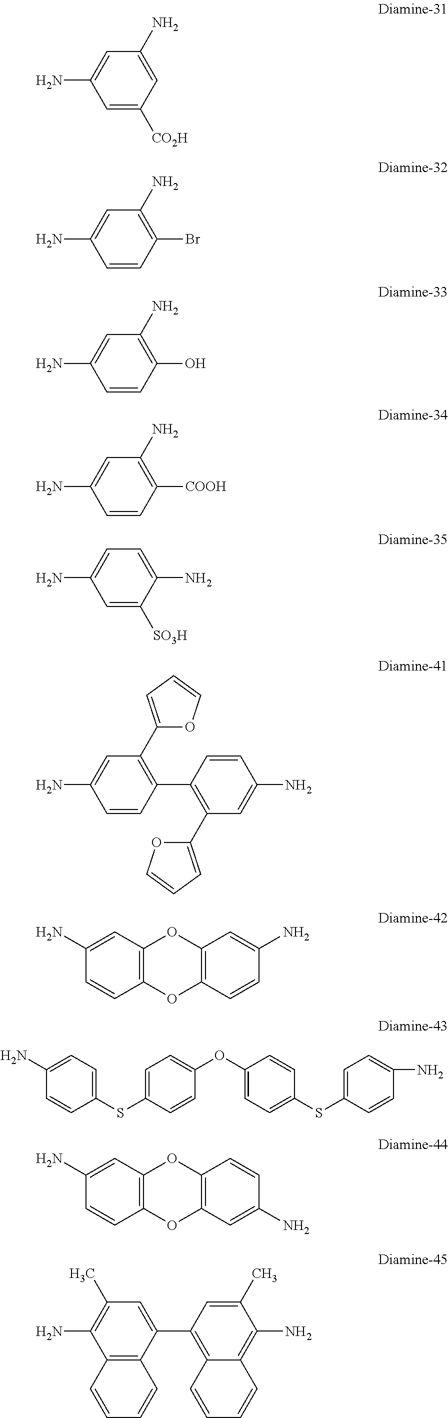

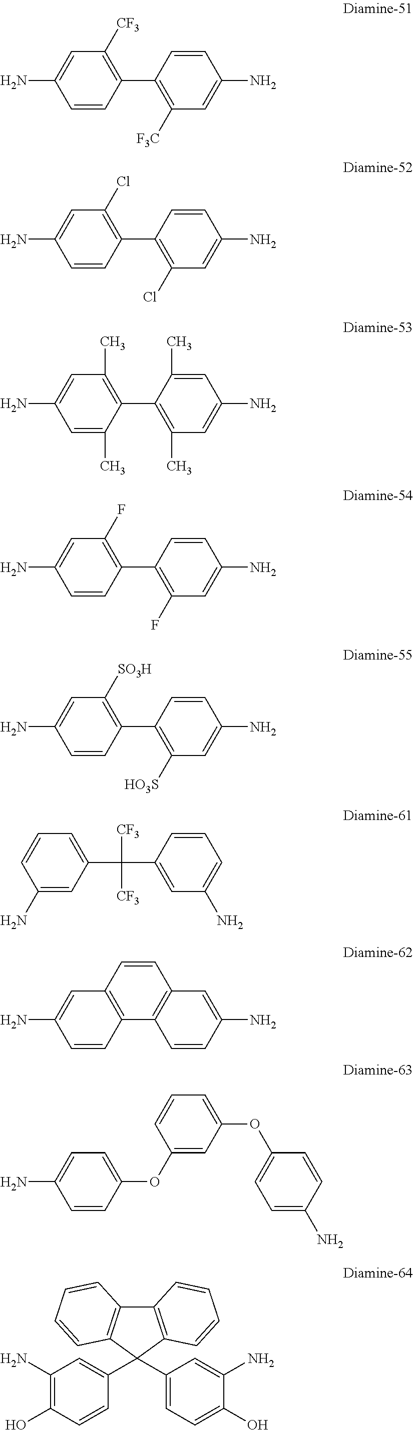

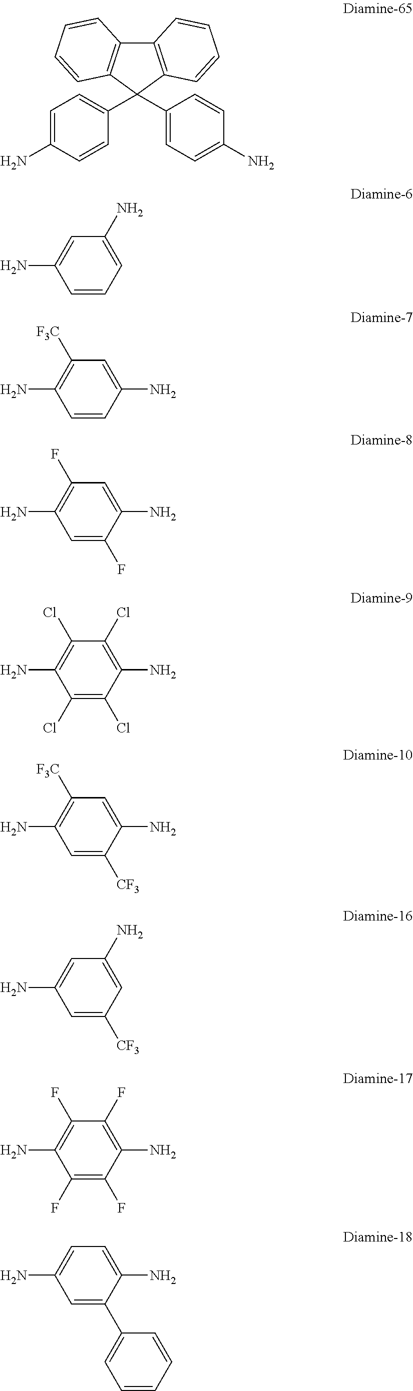

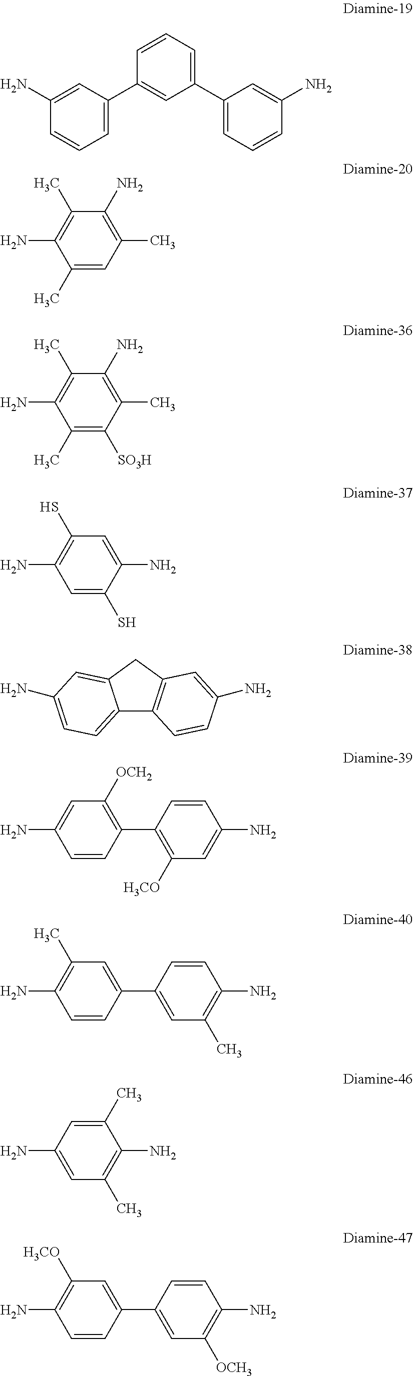

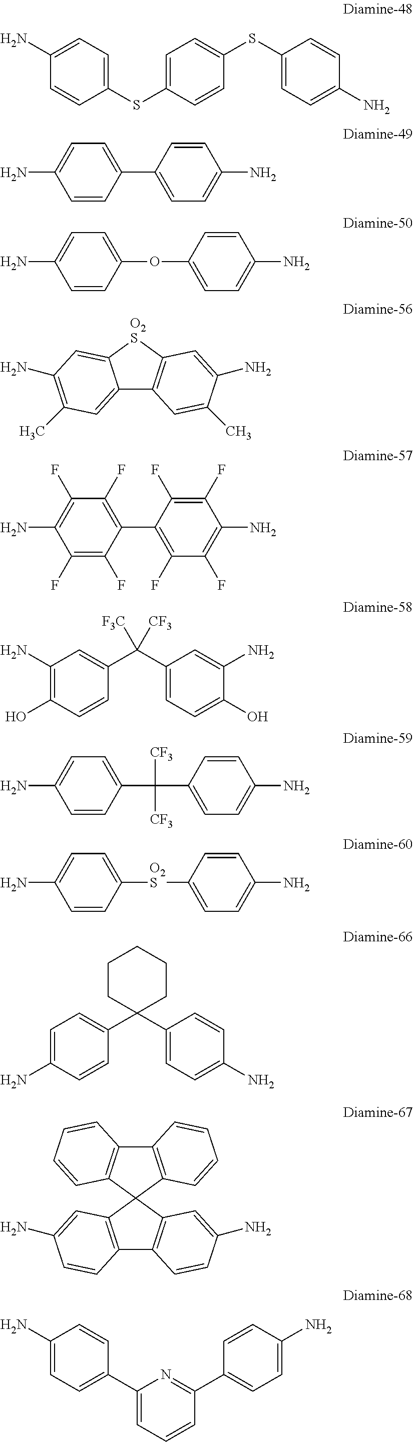

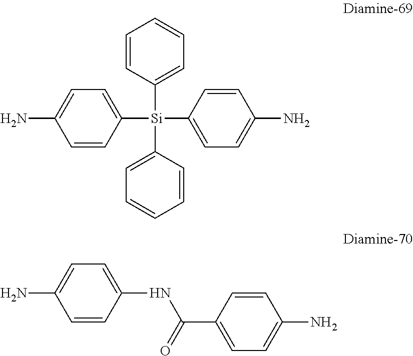 [SMILES: Nc1ccc([Si](c2ccccc2)(c2ccccc2)c2ccc(N)cc2)cc1 ; Nc1ccc(NC(=O)c2ccc(N)cc2)cc1]

The monomer represented by Formula (IV) or Formulae (IV-1) to (IV-3) and the monomer represented by Formulae (V-1) to (V-3), (VII-a), or (VII-b) are allowed to react with each other in advance to prepare an oligomer or a prepolymer and then used. The polyimide compound used in the present invention may be any of a block copolymer, a random copolymer, and a graft copolymer.

The polyimide compound used in the present invention can be obtained by mixing the above-described raw materials in a solvent and condensing and polymerizing the mixture using a typical method as described above.

The solvent is not particularly limited, and examples thereof include an ester-based organic solvent such as methyl acetate, ethyl acetate, or butyl acetate; an aliphatic ketone such as acetone, methyl ethyl ketone, methyl isobutyl ketone, diacetone alcohol, cyclopentanone, or cyclohexanone; an ether-based organic solvent such as ethylene glycol dimethyl ether, dibutyl butyl ether, tetrahydrofuran, methyl cyclopentyl ether, or dioxane; an amide-based organic solvent such as N-methylpyrrolidone, 2-pyrrolidone, dimethylformamide, dimethylimidazolidinone, or dimethylacetamide; and a sulfur-containing organic solvent such as dimethyl sulfoxide or sulfolane. These organic solvents can be suitably selected within the range in which a tetracarboxylic dianhydride serving as a reaction substrate, a diamine compound, polyamic acid which is a reaction intermediate, and a polyimide compound which is a final product can be dissolved. Among these, an ester (preferably butyl acetate), an aliphatic ketone (preferably methyl ethyl ketone, methyl isobutyl ketone, diacetone alcohol, cyclopentanone, or cyclohexanone), an ether (diethylene glycol monomethyl ether or methyl cyclopentyl ether), an amide (preferably N-methylpyrrolidone), or a sulfur (dimethyl sulfoxide or sulfolane) is preferable. In addition, these can be used alone or in combination of two or more kinds thereof.

The temperature of the polymerization reaction is not particularly limited and a temperature which can be typically employed for the synthesis of the polyimide compound can be employed. Specifically, the temperature is preferably in a range of −40° C. to 200° C. and more preferably in a range of 100° C. to 200° C.

The polyimide compound can be obtained by imidizing the polyamic acid, which is generated by the above-described polymerization reaction, through a dehydration ring-closure reaction in a molecule. The method of the dehydration ring-closure can be performed by referring to the method described in a general book (for example, “The Latest Polyimide ˜Fundamentals and Applications˜” edited by Toshio Imai and Rikio Yokota, NTS Inc., Aug. 25, 2010, pp. 3 to 49). A thermal imidization method of performing heating in a temperature range of 120° C. to 200° C. and removing water generated as a by-product to the outside of the system for a reaction or a so-called chemical imidization method in which a dehydration condensation agent such as an acetic anhydride, dicyclohexylcarbodiimide, or triphenyl phosphite is used in the coexistence of a basic catalyst such as pyridine, triethylamine, or DBU is suitably used.

In the present invention, the total concentration of the tetracarboxylic dianhydride and the diamine compound in the polymerization reaction solution of the polyimide compound is not particularly limited, but is preferably in a range of 5% to 70% by mass, more preferably in a range of 5% to 50% by mass, and still more preferably in a range of 5% to 30% by mass.

It is preferable that the gas separation membrane containing the polyimide compound of the present invention has a toluene swelling ratio of 35% or less. Here, in a case where a polyimide single membrane formed of a polyimide compound is exposed to saturated toluene vapor, the toluene swelling ratio is an increase ratio of the weight of the exposed polyimide single membrane to the weight of the polyimide single membrane before the exposure and is measured according to the method described in the example below.

From the viewpoint of the plasticity resistance, the toluene swelling ratio thereof is more preferably less than 20% and still more preferably less than 10%.

[Gas Separation Membrane]

(Gas Separation Composite Membrane)

It is preferable that the gas separation composite membrane which is a preferred embodiment of the gas separation membrane of the present invention includes a gas permeating support layer and a gas separation layer containing a specific polyimide compound, and the gas separation layer is formed on the upper side of the support layer. It is preferable that this composite membrane is formed by coating (in the present specification, the concept “coating” includes the form of adhesion to a surface through immersion) at least a surface of a porous support with a coating solution (dope) to form the gas separation layer.

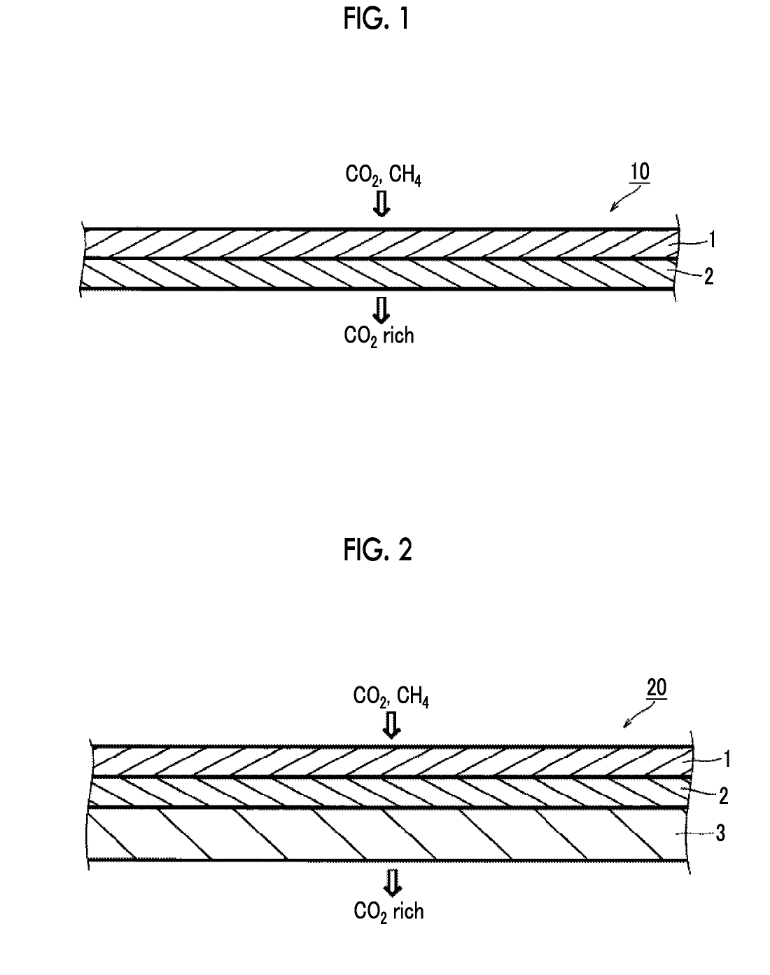

FIG. 1 is a longitudinal cross-sectional view schematically illustrating a gas separation composite membrane 10 which is a preferred embodiment of the present invention. The reference numeral 1 represents a gas separation layer and the reference numeral 2 represents a support layer formed of a porous layer. FIG. 2 is a cross-sectional view schematically illustrating a gas separation composite membrane 20 which is a preferred embodiment of the present invention. In the embodiment, a non-woven fabric layer 3 is added as a support layer in addition to the gas separation layer 1 and the porous layer 2.

FIGS. 1 and 2 illustrate the form of making permeating gas to be rich in carbon dioxide by selective permeation of carbon dioxide from a mixed gas of carbon dioxide and methane.

The expression “on the upper side of the support layer” in the present specification means that another layer may be interposed between the support layer and the gas separation layer. Further, in regard to the expressions related to up and down, the side where gas to be separated is supplied is set as “up” and the side where the separated gas is discharged is set as “down” unless otherwise specified.

The gas separation composite membrane of the present invention may be obtained by forming and disposing a gas separation layer on a surface or internal surface of the porous support (support layer) or can be obtained by simply forming a gas separation layer on at least a surface thereof to form a composite membrane. By forming a gas separation layer on at least a surface of the porous support, a composite membrane with an advantage of having excellent gas separation selectivity, excellent gas permeability, and mechanical strength can be obtained. As the membrane thickness of the gas separation layer, it is preferable that the gas separation layer is as thin as possible under conditions of imparting excellent gas permeability while maintaining the mechanical strength and the separation selectivity.

In the gas separation composite membrane of the present invention, the thickness of the gas separation layer is not particularly limited, but is preferably in a range of 0.01 to 5.0 μm and more preferably in a range of 0.05 to 2.0 μm.

The porous support (porous layer) which is preferably applied to the support layer is not particularly limited as long as the porous support is used for the purpose of imparting the mechanical strength and the excellent gas permeability, and the porous support may be formed of either of an organic material and an inorganic material. Among these, a porous membrane that contains an organic polymer is preferable. The thickness of the porous layer is preferably in a range of 1 to 3000 μm, more preferably in a range of 5 to 500 μm, and still more preferably in a range of 5 to 150 μm. The pore structure of this porous membrane has an average pore diameter of typically 10 μm or less, preferably 0.5 μm or less, and more preferably 0.2 μm or less. The porosity is preferably in a range of 20% to 90% and more preferably in a range of 30% to 80%.

Here, the support layer having the “gas permeability” means that the permeation rate of carbon dioxide is 1×10⁻⁵ cm³ (STP)/cm²·sec·cmHg (10 GPU) or greater in a case where carbon dioxide is supplied to the support layer (membrane formed of only the support layer) by setting the temperature to 40° C. and the total pressure on the side to which gas is supplied to 4 MPa. Further, in regard to the gas permeability of the support layer, the permeation rate of carbon dioxide is preferably 3×10⁻⁵ cm³ (STP)/cm²·sec·cmHg (30 GPU) or greater, more preferably 100 GPU or greater, and still more preferably 200 GPU or greater in a case where carbon dioxide is supplied by setting the temperature to 40° C. and the total pressure on the side to which gas is supplied to 4 MPa. STP stands for Standard Temperature and Pressure and GPU stands for Gas Permeation Unit. Examples of the material of the porous membrane include conventionally known polymers, for example, a polyolefin-based resin such as polyethylene or polypropylene; a fluorine-containing resin such as polytetrafluoroethylene, polyvinyl fluoride, or polyvinylidene fluoride; and various resins such as polystyrene, cellulose acetate, polyurethane, polyacrylonitrile, polyphenylene oxide, polysulfone, polyether sulfone, polyimide, and polyaramid. As the shape of the porous membrane, any shape from among a flat plate shape, a spiral shape, a tabular shape, and a hollow fiber shape can be employed.

In the gas separation composite membrane of the present invention, it is preferable that a support is formed in the lower portion of the support layer that forms the gas separation layer for imparting mechanical strength. Examples of such a support include woven fabric, non-woven fabric, and a net. Among these, from the viewpoints of membrane forming properties and the cost, non-woven fabric is suitably used. As the non-woven fabric, fibers formed of polyester, polypropylene, polyacrylonitrile, polyethylene, and polyamide may be used alone or in combination of plural kinds thereof. The non-woven fabric can be produced by papermaking main fibers and binder fibers which are uniformly dispersed in water using a circular net or a long net and then drying the fibers with a dryer. Moreover, for the purpose of removing a nap or improving mechanical properties, it is preferable that thermal pressing processing is performed on the non-woven fabric by interposing the non-woven fabric between two rolls.

<Method of Producing Gas Separation Composite Membrane>

As a method of producing the composite membrane of the present invention, a production method which includes coating a support with a coating solution containing the above-described polyimide compound to form a gas separation layer is preferable. The content of the polyimide compound in the coating solution is not particularly limited, but is preferably in a range of 0.1% to 30% by mass and more preferably in a range of 0.5% to 10% by mass. In a case where the content of the polyimide compound is extremely small, defects are highly likely to occur in the surface layer contributing to gas separation because the coating solution easily permeates to the underlayer at the time of formation of a film on the porous support. In addition, in a case where the content of the polyimide compound is extremely large, there is a possibility that the gas permeability is degraded because holes are filled with the coating solution at a high concentration at the time of formation of a film on the porous support. The gas separation membrane of the present invention can be appropriately produced by adjusting the molecular weight of the polymer, the structure, and the composition of the gas separation layer and the viscosity of the solution.

The organic solvent serving as a medium of the coating solution is not particularly limited, and examples thereof include a hydrocarbon-based organic solvent such as n-hexane or n-heptane; an ester-based organic solvent such as methyl acetate, ethyl acetate, or butyl acetate; lower alcohol such as methanol, ethanol, n-propanol, isopropanol, n-butanol, isobutanol, or tert-butanol; an aliphatic ketone such as acetone, methyl ethyl ketone, methyl isobutyl ketone, diacetone alcohol, cyclopentanone, or cyclohexanone; an ether-based organic solvent such as ethylene glycol, diethylene glycol, triethylene glycol, glycerin, propylene glycol, ethylene glycol monomethyl ether, ethylene glycol monoethyl ether, propylene glycol methyl ether, dipropylene glycol methyl ether, tripropylene glycol methyl ether, ethylene glycol phenyl ether, propylene glycol phenyl ether, diethylene glycol monomethyl ether, diethylene glycol monoethyl ether, diethylene glycol monobutyl ether, triethylene glycol monomethyl ether, triethylene glycol monoethyl ether, dibutyl butyl ether, tetrahydrofuran, methyl cyclopentyl ether, or dioxane; and N-methylpyrrolidone, 2-pyrrolidone, dimethylformamide, dimethylimidazolidinone, dimethyl sulfoxide, and dimethyl acetamide. These organic solvents are appropriately selected within the range that does not adversely affect the support through erosion or the like, and an ester (preferably butyl acetate), an alcohol (preferably methanol, ethanol, isopropanol, or isobutanol), an aliphatic ketone (preferably methyl ethyl ketone, methyl isobutyl ketone, diacetone alcohol, cyclopentanone, or cyclohexanone), and an ether (ethylene glycol, diethylene glycol monomethyl ether, or methyl cyclopentyl ether) are preferable and an aliphatic ketone, an alcohol, and an ether are more preferable. Further, these may be used alone or in combination of two or more kinds thereof.

<Another Layer Between Support Layer and Gas Separation Layer>

In the gas separation composite membrane of the present invention, another layer may be present between the support layer and the gas separation layer. Preferred examples of another layer include a siloxane compound layer. By providing a siloxane compound layer, unevenness of the outermost surface of the support layer can be made to be smooth and the thickness of the gas separation layer is easily reduced. Examples of a siloxane compound that forms the siloxane compound layer include a compound in which the main chain is formed of polysiloxane and a compound having a siloxane structure and a non-siloxane structure in the main chain.

The “siloxane compound” in the present specification indicates an organopolysiloxane compound unless otherwise noted.

—Siloxane Compound Whose Main Chain is Formed of Polysiloxane—

As the siloxane compound which can be used for the siloxane compound layer and whose main chain is formed of polysiloxane, one or two or more kinds of polyorganopolysiloxanes represented by Formula (1) or (2) may be exemplified. Further, these polyorganopolysiloxanes may form a crosslinking reactant. As the crosslinking reactant, a compound in the form of the compound represented by Formula (1) being crosslinked by a polysiloxane compound having groups linked to each other by reacting with a reactive group X^{S} of Formula (1) at both terminals is exemplified.

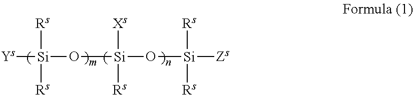

In Formula (1), R^{S} represents a non-reactive group. Specifically, it is preferable that R^{S} represents an alkyl group (an alkyl group having preferably 1 to 18 carbon atoms and more preferably 1 to 12 carbon atoms) or an aryl group (an aryl group having preferably 6 to 15 carbon atoms and more preferably 6 to 12 carbon atoms; and more preferably phenyl).

X^{S} represents a reactive group, and it is preferable that X^{S} represents a group selected from a hydrogen atom, a halogen atom, a vinyl group, a hydroxyl group, and a substituted alkyl group (an alkyl group having preferably 1 to 18 carbon atoms and more preferably 1 to 12 carbon atoms).

Y^{S} and Z^{S} are the same as R^{S} or X^{S} described above.

m represents a number of 1 or greater and preferably 1 to 100,000.

n represents a number of 0 or greater and preferably 0 to 100,000.

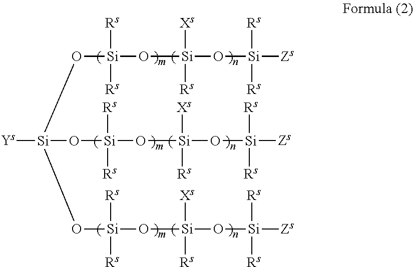

In Formula (2), X^{S}, Y^{S}, Z^{S}, R^{S}, m, and n each have the same definition as that for X^{S}, Y^{S}, Z^{S}, R^{S}, m, and n in Formula (1).

In Formulae (1) and (2), in a case where the non-reactive group R^{S} represents an alkyl group, examples of the alkyl group include methyl, ethyl, hexyl, octyl, decyl, and octadecyl. Further, in a case where the non-reactive group R^{S} represents a fluoroalkyl group, examples of the fluoroalkyl group include —CH₂CH₂CF₃, and —CH₂CH₂C₆F₁₃.

In Formulae (1) and (2), in a case where the reactive group X^{S} represents a substituted alkyl group, examples of the alkyl group include a hydroxyalkyl group having 1 to 18 carbon atoms, an aminoalkyl group having 1 to 18 carbon atoms, a carboxyalkyl group having 1 to 18 carbon atoms, a cycloalkyl group having 1 to 18 carbon atoms, a glycidoxyalkyl group having 1 to 18 carbon atoms, a glycidyl group, an epoxycyclohexylalkyl group having 7 to 16 carbon atoms, a (1-oxacyclobutane-3-yl)alkyl group having 4 to 18 carbon atoms, a methacryloxyalkyl group, and a mercaptoalkyl group.

The number of carbon atoms of the alkyl group constituting the hydroxyalkyl group is preferably an integer of 1 to 10, and examples of the hydroxyalkyl group include —CH₂CH₂CH₂OH.

The number of carbon atoms of the alkyl group constituting the aminoalkyl group is preferably an integer of 1 to 10, and examples of the aminoalkyl group include —CH₂CH₂CH₂NH₂.

The number of carbon atoms of the alkyl group constituting the carboxyalkyl group is preferably an integer of 1 to 10, and examples of the carboxyalkyl group include —CH₂CH₂CH₂COOH.

The number of carbon atoms of the alkyl group constituting the chloroalkyl group is preferably an integer of 1 to 10, and preferred examples of the chloroalkyl group include —CH₂Cl.

The number of carbon atoms of the alkyl group constituting the glycidoxyalkyl group is preferably an integer of 1 to 10, and preferred examples of the glycidoxyalkyl group include 3-glycidyloxypropyl.

The number of carbon atoms of the epoxycyclohexylalkyl group having 7 to 16 carbon atoms is preferably an integer of 8 to 12.

The number of carbon atoms of the (1-oxacyclobutane-3-yl)alkyl group having 4 to 18 carbon atoms is preferably an integer of 4 to 10.

The number of carbon atoms of the alkyl group constituting the methacryloxyalkyl group is preferably an integer of 1 to 10, and examples of the methacryloxyalkyl group include —CH₂CH₂CH₂—OOC—C(CH₃)═CH₂.

The number of carbon atoms of the alkyl group constituting the mercaptoalkyl group is preferably an integer of 1 to 10, and examples of the mercaptoalkyl group include —CH₂CH₂CH₂SH.

It is preferable that m and n represent a number in which the molecular weight of the compound is in a range of 5,000 to 1000,000.

In Formulae (1) and (2), distribution of a reactive group-containing siloxane unit (in the formulae, a constitutional unit whose number is represented by n) and a siloxane unit (in the formulae, a constitutional unit whose number is represented by m) which does not have a reactive group is not particularly limited. That is, in Formulae (1) and (2), the (Si(R^{S})(R^{S})—O) unit and the (Si(R^{S})(X^{S})—O) unit may be randomly distributed.

—Compound Having Siloxane Structure and Non-Siloxane Structure in Main Chain—

Examples of the compound which can be used for the siloxane compound layer and has a siloxane structure and a non-siloxane structure in the main chain include compounds represented by Formulae (3) to (7).

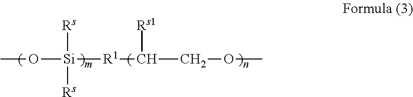

In Formula (3), R^{S}, m, and n each have the same definition as that for R^{S}, m, and n in Formula (1). R^{L} represents —O— or —CH₂— and R^{S1} represents a hydrogen atom or methyl. It is preferable that both terminals of Formula (3) are formed of an amino group, a hydroxyl group, a carboxy group, a trimethylsilyl group, an epoxy group, a vinyl group, a hydrogen atom, or a substituted alkyl group.

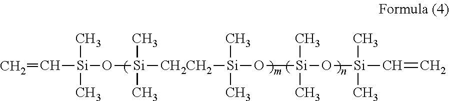

In Formula (4), m and n each have the same definition as that for m and n in Formula (1).

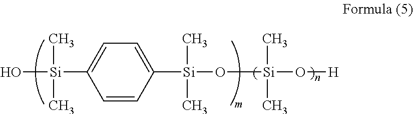

In Formula (5), m and n each have the same definition as that for m and n in Formula (1).

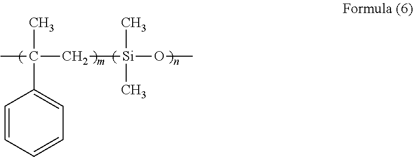

In Formula (6), m and n each have the same definition as that for m and n in Formula (1). It is preferable that both terminals of Formula (6) are bonded to an amino group, a hydroxyl group, a carboxy group, a trimethylsilyl group, an epoxy group, a vinyl group, a hydrogen atom, or a substituted alkyl group.

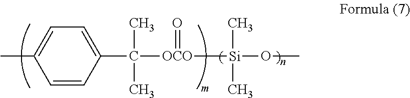

In Formula (7), m and n each have the same definition as that for m and n in Formula (1). It is preferable that both terminals of Formula (7) are bonded to an amino group, a hydroxyl group, a carboxy group, a trimethylsilyl group, epoxy, a vinyl group, a hydrogen atom, or a substituted alkyl group.

In Formulae (3) to (7), distribution of a siloxane structural unit and a non-siloxane structural unit may be randomly distributed.

It is preferable that the compound having a siloxane structure and a non-siloxane structure in the main chain contains 50% by mole or greater of the siloxane structural unit and more preferable that the compound contains 70% by mole or greater of the siloxane structural unit with respect to the total molar amount of all repeating structural units.

From the viewpoint of achieving the balance between durability and reduction in membrane thickness, the weight-average molecular weight of the siloxane compound used for the siloxane compound layer is preferably in a range of 5,000 to 1,000,000. The method of measuring the weight-average molecular weight is as described above.

Further, preferred examples of the siloxane compound constituting the siloxane compound layer are as follows.

Preferred examples thereof include one or two or more selected from polydimethylsiloxane, polymethylphenylsiloxane, polydiphenylsiloxane, a polysulfone-polyhydroxystyrene-polydimethylsiloxane copolymer, a dimethyl siloxane-methylvinylsiloxane copolymer, a dimethylsiloxane-diphenylsiloxane-methylvinylsiloxane copolymer, a methyl-3,3,3-trifluoropropylsiloxane-methylvinylsiloxane copolymer, a dimethylsiloxane-methylphenylsiloxane-methylvinylsiloxane copolymer, a vinyl terminated diphenylsiloxane-dimethyl siloxane copolymer, vinyl terminated polydimethylsiloxane, H terminated polydimethylsiloxane, and a dimethyl siloxane-methylhydroxysiloxane copolymer. Further, these compounds also include the forms of forming crosslinking reactants.

In the composite membrane of the present invention, from the viewpoints of smoothness and gas permeability, the thickness of the siloxane compound layer is preferably in a range of 0.01 to 5 μm and more preferably in a range of 0.05 to 1 μm.

Further, the gas permeability of the siloxane compound layer at 40° C. and 4 MPa is preferably 100 GPU or greater, more preferably 300 GPU or greater, and still more preferably 1000 GPU or greater in terms of the permeation rate of carbon dioxide.

(Gas Separation Asymmetric Membrane)

The gas separation membrane of the present invention may be an asymmetric membrane. The asymmetric membrane can be formed according to a phase inversion method using a solution containing a polyimide compound. The phase inversion method is a known method of allowing a polymer solution to be brought into contact with a coagulating liquid for phase inversion to form a membrane, and a so-called dry-wet method is suitably used in the present invention. The dry-wet method is a method of forming a porous layer by evaporating a solution on the surface of a polymer solution which is made to have a membrane shape to form a thin compact layer, immersing the compact layer in a coagulating liquid (the coagulating liquid is a solvent which is compatible with a solvent of a polymer solution and in which a polymer is insoluble), and forming fine pores using a phase separation phenomenon that occurs at this time, and this method is suggested by Loeb and Sourirajan et al. (for example, the specification of U.S. Pat. No. 3,133,132A).

In the gas separation asymmetric membrane of the present invention, the thickness of the surface layer contributing to gas separation, which is referred to as a compact layer or a skin layer, is not particularly limited, but is preferably in a range of 0.01 to 5.0 μm and more preferably in a range of 0.05 to 1.0 μm from the viewpoint of imparting practical gas permeability. In addition, the porous layer positioned in the lower portion of the compact layer plays a role of decreasing gas permeability resistance and imparting the mechanical strength at the same time, and the thickness thereof is not particularly limited as long as self-supporting properties as an asymmetric membrane are imparted, but is preferably in a range of 5 to 500 μm, more preferably in a range of 5 to 200 μm, and still more preferably in a range of 5 to 100 μm.

The gas separation asymmetric membrane of the present invention may be a flat membrane or a hollow fiber membrane. An asymmetric hollow fiber membrane can be produced by a dry-wet spinning method. The dry-wet spinning method is a method of producing an asymmetric hollow fiber membrane by applying a dry-wet method to a polymer solution which is discharged from a spinning nozzle in a target shape which is a hollow fiber shape. More specifically, the dry-wet spinning method is a method in which a polymer solution is discharged from a nozzle in a target shape which is a hollow fiber shape and passes through air or a nitrogen gas atmosphere immediately after the discharge. Thereafter, an asymmetric structure is formed through immersion in a coagulating liquid which does not substantially dissolve a polymer and is compatible with a solvent of the polymer solution. Further, the membrane is dried and subjected to a heat treatment as necessary, thereby producing a separation membrane.

It is preferable that the solution viscosity of the solution containing a polyimide compound which is discharged from a nozzle is in a range of 2 to 17000 Pa·s, preferably 10 to 1500 Pa·s, and particularly preferably in a range of 20 to 1000 Pa·s at the discharge temperature (for example, 10° C.) from a viewpoint of stably obtaining the shape after the discharge such as a hollow fiber shape or the like. It is preferable that immersion of a membrane in a coagulating liquid is carried out by immersing the membrane in a primary coagulating liquid to be solidified to the extent that the shape of a membrane such as a hollow fiber shape can be maintained, winding the membrane around a guide roll, immersing the membrane in a secondary coagulating liquid, and sufficiently solidifying the whole membrane. It is efficient that the solidified membrane is dried after the coagulating liquid is substituted with a solvent such as hydrocarbon. It is preferable that the heat treatment for drying the membrane is performed at a temperature lower than the softening point or the secondary transition point of the used polyimide compound.

(Use and Properties of Gas Separation Membrane)

The gas separation membrane (the composite membrane and the asymmetric membrane) of the present invention can be suitably used according to a gas separation recovery method and a gas separation purification method. For example, a gas separation membrane which is capable of efficiently separating specific gas from a gas mixture containing gas, for example, hydrocarbon such as hydrogen, helium, carbon monoxide, carbon dioxide, hydrogen sulfide, oxygen, nitrogen, ammonia, a sulfur oxide, a nitrogen oxide, methane, or ethane; unsaturated hydrocarbon such as propylene; or a perfluoro compound such as tetrafluoroethane can be obtained. Particularly, it is preferable that a gas separation membrane selectively separating carbon dioxide from a gas mixture containing carbon dioxide and hydrocarbon (methane) is obtained.

In addition, in a case where gas subjected to a separation treatment is a mixed gas of carbon dioxide and methane, the permeation rate of the carbon dioxide at 30° C. and 5 MPa is preferably greater than 20 GPU, more preferably greater than 30 GPU, and still more preferably in a range of 35 GPU to 500 GPU. The ratio between permeation rates of carbon dioxide and methane (R_{CO2}a_{CH4}) is preferably 15 or greater, and more preferably 20 or greater. R_{CO2} represents the permeation rate of carbon dioxide and R_{CH4} represents the permeation rate of methane.

Further, 1 GPU is 1×10⁻⁶ cm³ (STP)/cm²·cm·sec·cmHg.

### Other Components and the Like

Various polymer compounds can also be added to the gas separation layer of the gas separation membrane of the present invention in order to adjust the physical properties of the membrane. As the polymer compounds, an acrylic polymer, a polyurethane resin, a polyamide resin, a polyester resin, an epoxy resin, a phenol resin, a polycarbonate resin, a polyvinyl butyral resin, a polyvinyl formal resin, shellac, a vinyl-based resin, an acrylic resin, a rubber-based resin, waxes, and other natural resins can be used. Further, these may be used in combination of two or more kinds thereof.

Further, a non-ionic surfactant, a cationic surfactant, or an organic fluoro compound can be added to the gas separation membrane of the present invention in order to adjust the physical properties of the liquid.

Specific examples of the surfactant include anionic surfactants such as alkyl benzene sulfonate, alkyl naphthalene sulfonate, higher fatty acid salts, sulfonate of higher fatty acid ester, sulfuric ester salts of higher alcohol ether, sulfonate of higher alcohol ether, alkyl carboxylate of higher alkyl sulfonamide, and alkyl phosphate; non-ionic surfactants such as polyoxyethylene alkyl ether, polyoxyethylene alkyl phenyl ether, polyoxyethylene fatty acid ester, sorbitan fatty acid ester, an ethylene oxide adduct of acetylene glycol, an ethylene oxide adduct of glycerin, and polyoxyethylene sorbitan fatty acid ester; and amphoteric surfactants such as alkyl betaine and amide betaine; a silicon-based surfactant; and a fluorine-based surfactant, and the surfactant can be suitably selected from known surfactants and derivatives thereof in the related art.

Further, a polymer dispersant may be included, and specific examples of the polymer dispersant include polyvinyl pyrrolidone, polyvinyl alcohol, polyvinyl methyl ether, polyethylene oxide, polyethylene glycol, polypropylene glycol, and polyacrylamide. Among these, polyvinyl pyrrolidone is preferably used.

The conditions of forming the gas separation membrane of the present invention are not particularly limited. The temperature thereof is preferably in a range of −30° C. to 100° C., more preferably in a range of −10° C. to 80° C., and particularly preferably in a range of 5° C. to 50° C.

In the present invention, gas such as air or oxygen may be allowed to coexist during membrane formation, and it is desired that the membrane is formed under an inert gas atmosphere.

In the gas separation membrane of the present invention, the content of the polyimide compound in the gas separation layer is not particularly limited as long as desired gas separation performance can be obtained. From the viewpoint of further improving gas separation performance, the content of the polyimide compound in the gas separation layer is preferably 20% by mass or greater, more preferably 40% by mass or greater, still more preferably 60% by mass or greater, and even still more preferably 70% by mass or greater. Further, the content of the polyimide compound in the gas separation layer may be 100% by mass and is typically 99% by mass or less.

[Method of Separating Gas Mixture]

The gas separation method of the present invention is a method that includes selectively permeating carbon dioxide from a mixed gas containing carbon dioxide and methane. The gas pressure at the time of gas separation is preferably in a range of 0.5 MPa to 10 MPa, more preferably in a range of 1 MPa to 10 MPa, and still more preferably in a range of 2 MPa to 7 MPa. Further, the temperature for separating gas is preferably in a range of −30° C. to 90° C. and more preferably in a range of 15° C. to 70° C. In the mixed gas containing carbon dioxide and methane gas, the mixing ratio of carbon dioxide to methane gas is not particularly limited. The mixing ratio thereof (carbon dioxide:methane gas) is preferably in a range of 1:99 to 99:1 (volume ratio) and more preferably in a range of 5:95 to 90:10.

[Gas Separation Module and Gas Separator]

A gas separation membrane module can be prepared using the gas separation membrane of the present invention. Examples of the module include a spiral type module, a hollow fiber type module, a pleated module, a tubular module, and a plate and frame type module.

Moreover, it is possible to obtain a gas separator having means for performing separation and recovery of gas or performing separation and purification of gas by using the gas separation composite membrane of the present invention or the gas separation membrane module. The gas separation composite membrane of the present invention may be applied to a gas separation and recovery device which is used together with an absorption liquid described in JP2007-297605A according to a membrane/absorption hybrid method.

### EXAMPLES

Hereinafter, the present invention will be described in detail with reference to examples, but the present invention is not limited these examples.

### Synthesis Example

<Synthesis of Polyimide Compound (P-101)>

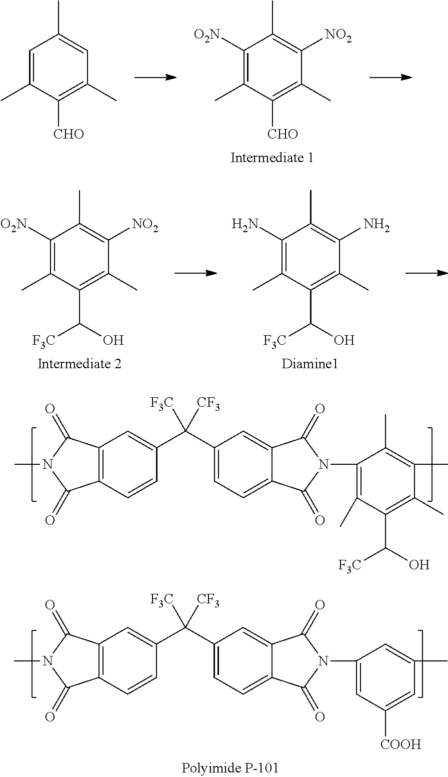

(Synthesis of Intermediate 1)

Sulfuric acid (manufactured by Wako Pure Chemical Industries, Ltd.) (100 ml) was added to a 1 L flask, nitric acid (1.42 g/ml, manufactured by Wako Pure Chemical Industries, Ltd.) (100 ml) was carefully added dropwise thereto under an ice cooling condition, and then 2,4,6-trimethylbenzaldehyde (manufactured by Tokyo Chemical Industry Co., Ltd.) (22.5 g) was carefully added dropwise thereto under an ice cooling condition for a reaction at room temperature for 6 hours. The reaction solution was poured into ice water and purified, thereby obtaining an intermediate 1 (35 g).

(Synthesis of Intermediate 2)

Tetrahydrofuran (manufactured by Wako Pure Chemical Industries, Ltd.) (25 mL) and the intermediate 1 (3 g) were added to a 100 mL flask. Tetrabutylammonium fluoride (1 mol/L tetrahydrofuran solution, manufactured by Tokyo Chemical Industry Co., Ltd.) (0.3 g) was carefully added dropwise thereto under an ice cooling condition, and trifluoromethyl trimethylsilane (manufactured by Tokyo Chemical Industry Co., Ltd.) (2 g) was carefully added dropwise thereto under an ice cooling condition for a reaction at room temperature for 1 hour. Next, hydrochloric acid (manufactured by Wako Pure Chemical Industries, Ltd.) (25 mL) was added to the reaction solution, and the reaction solution was allowed to react at room temperature for 5 hours. The reaction solution was concentrated under reduced pressure and purified using silica gel column chromatography, thereby obtaining an intermediate 2 (3 g).

(Synthesis of Diamine 1)

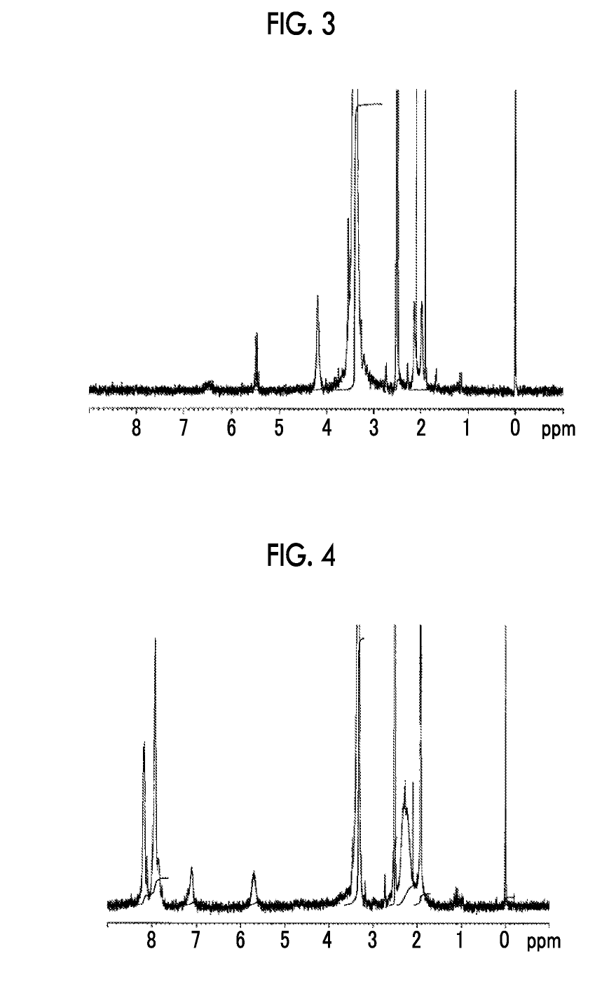

Reduced iron (manufactured by Wako Pure Chemical Industries, Ltd.) (4 g), ammonium chloride (manufactured by Wako Pure Chemical Industries, Ltd.) (0.4 g), isopropanol (manufactured by Wako Pure Chemical Industries, Ltd.) (20 mL), and water (5 mL) were added to a 100 mL flask and heated and refluxed for 10 minutes. Acetic acid (manufactured by Wako Pure Chemical Industries, Ltd.) (0.4 mL) and the intermediate 2 (3 g) were added to the solution after the reflux and further heated and refluxed for 30 minutes. The reaction solution was concentrated under reduced pressure and purified using silica gel chromatography, thereby obtaining a diamine 1 (1 g). The results of ¹H-NMR (deuterated solvent: dimethyl sulfoxide (DMSO)-d6) of the diamine 1 are shown in FIG. 3.

(Synthesis of Polyimide Compound (P-101))

N-methylpyrrolidone (manufactured by Wako Pure Chemical Industries, Ltd.) (20 g), the diamine 1 (1.361 g), DABA (3,5-diaminobenzoic acid) (manufactured by Nipponjunryo Chemicals) (0.0927 g), and 6FDA (4,4′-(hexafluoroisopropylidene)diphthalic anhydride) (manufactured by Tokyo Chemical Industry Co., Ltd.) (2.705 g) were added to a 100 mL flask. Toluene (manufactured by Wako Pure Chemical Industries, Ltd.) (5 g) was added thereto, and the solution was heated to 180° C. for a reaction for 6 hours. After the reaction solution was cooled, the reaction solution was diluted with acetone (manufactured by Wako Pure Chemical Industries, Ltd.). Methanol (manufactured by Wako Pure Chemical Industries, Ltd.) was added to the diluted solution to obtain a polymer as a solid. The same re-precipitation was repeated twice, and the resultant was dried at 80° C., thereby obtaining polyimide (P-101) (3 g).

The results of ¹H-NMR (deuterated solvent: DMSO-d6) of the polyimide compound (P-101) are shown in FIG. 4.

A content A of the structure represented by Formula (1), contained in the polyimide compound (P-101), was acquired in the following manner.

100 mg of benzotrifluoride (manufactured by Tokyo Chemical Industry Co., Ltd.) was dissolved in 100 ml of a deuterated solvent DMSO-d6 (manufactured by Tokyo Chemical Industry Co., Ltd., 99.9 atom % D) to obtain an NMR solvent. 10 mg of the polyimide compound (P-101) and 1 ml of the NMR solvent were mixed and stirred for 1 hour, and 300 MHz ¹⁹F-NMR was measured.

The CF₃ peak surface area of the benzotrifluoride was normalized as 1, the CF₃ peak (detected at approximately −70 to −80 ppm, but varies depending on the substituent) area in the structure represented by Formula (1) was set as S, and the content A was calculated according to the following equation.

S/146.11×100=A[mmol/g]

The content A of the structure represented by Formula (1), contained in the polyimide compound (P-101), was 1.39 mmol/g.

The content A of the structure represented by Formula (1) can be calculated from the peak surface area derived from an OH group in Formula (1) using ¹H-NMR.

The weight-average molecular weight of the obtained polyimide compound (P-101) was measured (flow rate: 0.35 ml/min, temperature: 40° C., eluent: THF) using an HLC-8220GPC device (manufactured by Tosoh Corporation), and the weight-average molecular weight thereof was 140,000.

—GPC Measurement Conditions—
Device: TOSOH HLC-8220
GPC column: TOSOH TSKgel Super HZM-H, TOSOH TSKgel Super HZ4000, TOSOH TSKgel, Super HZ2000
Eluent: containing THF stabilizer (206-05106, manufactured by Wako Pure Chemical Industries, Ltd.)
Flow rate: 0.35 ml/min
Temperature: 40° C.
Time for analysis: 20 minutes
Sampling pitch: 100 msec
Concentration: 0.1 wt %
Injection amount: 10 μl
Standard: TOSOH TSK standard POLYSTYRENE

The structure derived from a tetracarboxylic dianhydride and the structure derived from a diamine which constitute the polyimide compound (P-101) synthesized in the synthesis example are shown in Formula (P-100). The polyimide compound (P-101) is a polyimide compound formed by a, b, c, and d in Formula (P-100) respectively having the copolymerization ratio listed in Table 1. Similarly, various polyimide compounds to be synthesized in the present example have a unit structure formed by combining two or more structures derived from a tetracarboxylic dianhydride represented by a or b in the following formula and structures derived from a diamine represented by c or d in the following formula, and the copolymerization ratios thereof are listed Tables 1 and 2.

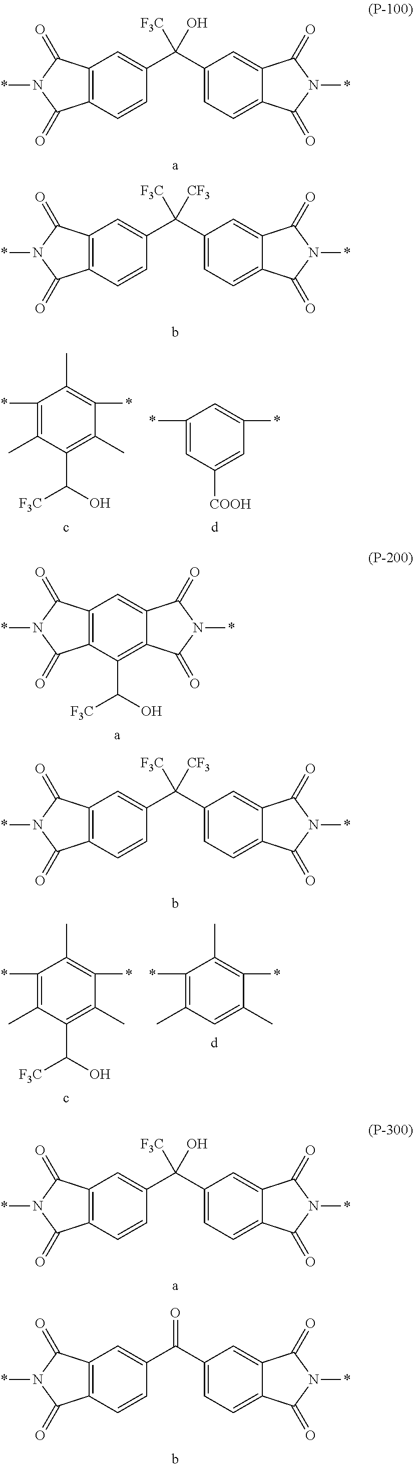

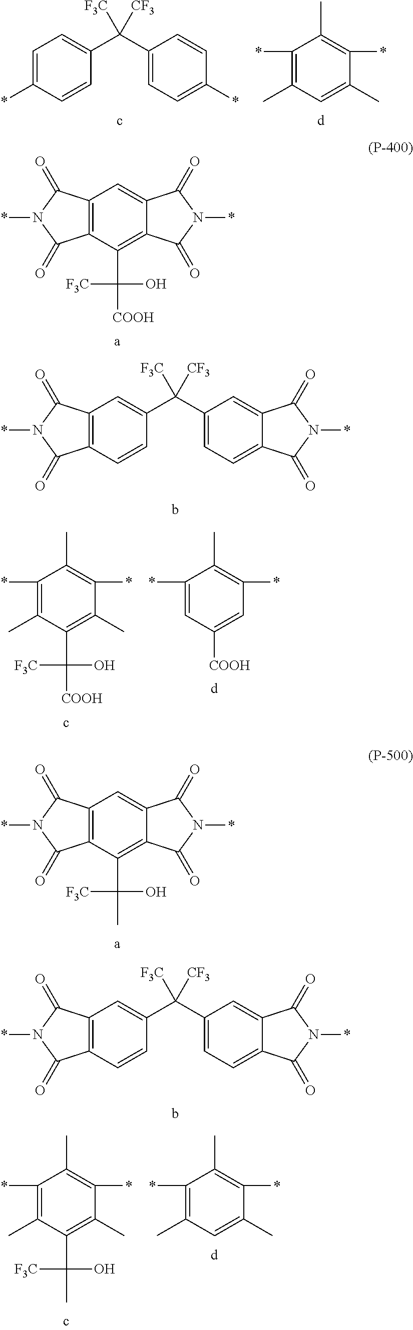

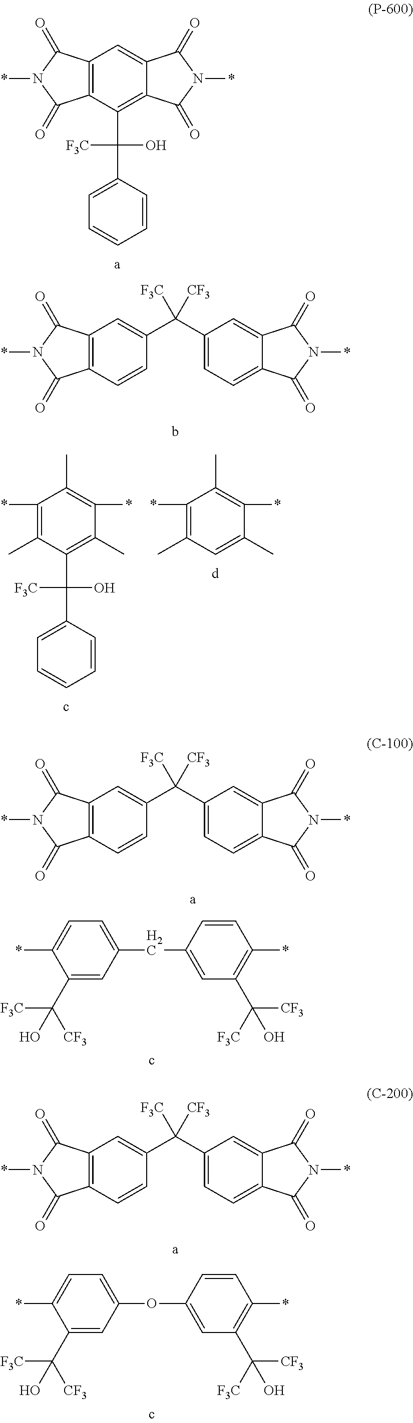

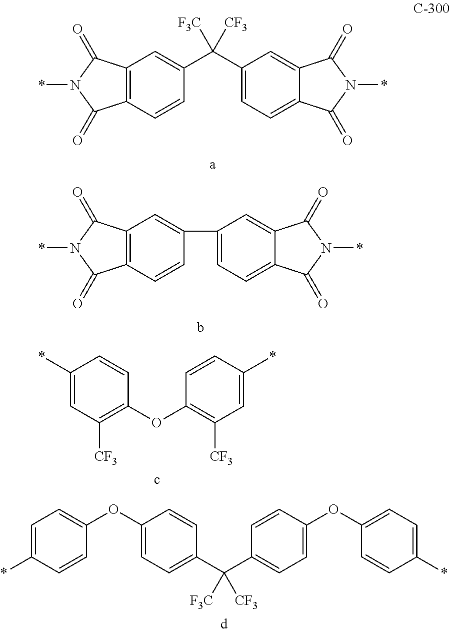

<Synthesis of Polyimide Compound (P-102)>

A polyimide compound (P-102) was obtained in the same manner as in the synthesis of the polyimide compound (P-101) except that the copolymerization ratios of a, b, c, and d components in Formula (P-100) were changed to the copolymerization ratios listed in Table 1.

<Synthesis of Polyimide Compounds (P-201) to (P-204)>

Polyimide compounds (P-201) to (P-204) were obtained in the same manner as in the synthesis of the polyimide compound (P-101) except that a, b, c, and d components in Formula (P-100) were changed to a, b, c, and d components in Formula (P-200) and the copolymerization ratios thereof were changed to the copolymerization ratios listed in Table 1.

<Synthesis of polyimide compounds (P-301) to (P-302)>

Polyimide compounds (P-301) and (P-302) were obtained in the same manner as in the synthesis of the polyimide compound (P-101) except that a, b, c, and d components in Formula (P-100) were changed to a, b, c, and d components in Formula (P-300) and the copolymerization ratios thereof were changed to the copolymerization ratios listed in Table 1.

<Synthesis of Polyimide Compounds (P-401) to (P-402)>

Polyimide compounds (P-401) and (P-402) were obtained in the same manner as in the synthesis of the polyimide compound (P-101) except that a, b, c, and d components in Formula (P-100) were changed to a, b, c, and d components in Formula (P-400) and the copolymerization ratios thereof were changed to the copolymerization ratios listed in Table 1.

<Synthesis of Polyimide Compounds (P-501) and (P-502)>

Polyimide compounds (P-501) and (P-502) were obtained in the same manner as in the synthesis of the polyimide compound (P-101) except that a, b, c, and d components in Formula (P-100) were changed to a, b, c, and d components in Formula (P-500) and the copolymerization ratios thereof were changed to the copolymerization ratios listed in Table 1.

<Synthesis of Polyimide Compounds (P-601) and (P-602)>

Polyimide compounds (P-601) and (P-602) were obtained in the same manner as in the synthesis of the polyimide compound (P-101) except that a, b, c, and d components in Formula (P-100) were changed to a, b, c, and d components in Formula (P-600) and the copolymerization ratios thereof were changed to the copolymerization ratios listed in Table 1.

TABLE 1

| | Copolymerization ratio | | | | | |
|---|---|---|---|---|---|---|
| | (molar ratio) | | | | | |
| | Acid | | | | | Weight-average |
| | dianhydride | | Diamine | | Content A | molecular |
| Polyimide | a | b | c | d | [mmol/g] | weight [×10³] |
| P-101 | 0 | 100 | 90 | 10 | 1.39 | 140 |
| P-102 | 100 | 0 | 100 | 0 | 3.31 | 103 |
| P-201 | 100 | 0 | 100 | 0 | 3.79 | 95 |
| P-202 | 0 | 100 | 100 | 0 | 1.52 | 110 |
| P-203 | 0 | 100 | 60 | 40 | 0.97 | 104 |
| P-204 | 0 | 100 | 20 | 80 | 0.35 | 77 |
| P-301 | 100 | 0 | 100 | 0 | 3.13 | 99 |
| P-302 | 100 | 0 | 0 | 100 | 1.97 | 131 |
| P-401 | 100 | 0 | 100 | 0 | 3.24 | 125 |
| P-402 | 50 | 50 | 50 | 50 | 1.70 | 122 |
| P-501 | 100 | 0 | 100 | 0 | 3.59 | 158 |
| P-502 | 50 | 50 | 50 | 50 | 1.79 | 196 |
| P-601 | 100 | 0 | 100 | 0 | 2.94 | 171 |
| P-602 | 50 | 50 | 50 | 50 | 1.61 | 85 |

<Synthesis of Polyimide Compound (C-101) and (C-201)>

Polyimide solutions were synthesized in the same manner as in the synthesis of the polyimide 1 and the polyimide 10 described in JP2013-10096A and diluted with acetone (manufactured by Wako Pure Chemical Industries, Ltd.), and methanol (manufactured by Wako Pure Chemical Industries, Ltd.) was added thereto to obtain a polymer as a solid. The same re-precipitation was respectively repeated twice, the resultants were dried at 80° C., and a polyimide compound (C-101) and a polyimide compound (C-201) were synthesized. The polyimide compound (C-101) is formed of a and b components shown in Formula (C-100) and the molar ratios of the a component and the b component are as listed in Table 2. Further, the polyimide compound (C-201) is formed of a and b components shown in Formula (C-200) and the molar ratios of the a component and the b component are as listed in Table 2.

<Synthesis of Polyimide Compound (C-301)>

A polyimide solution was synthesized in the same manner as in Example 1 described in JP2011-183370A and diluted with acetone (manufactured by Wako Pure Chemical Industries, Ltd.), and methanol (manufactured by Wako Pure Chemical Industries, Ltd.) was added thereto to obtain a polymer as a solid. The same re-precipitation was respectively repeated twice, the resultant was dried at 80° C., and a polyimide compound (C-301) was synthesized. The polyimide compound (C-301) is formed of a and b components shown in Formula (C-300) and the molar ratios of the a component and the b component are as listed in Table 2.

TABLE 2

| | Copolymerization ratio | | | | | |
|---|---|---|---|---|---|---|
| | (molar ratio) | | | | | |
| | Acid | | | | | Weight-average |
| | dianhydride | | Diamine | | Content A | molecular |
| Polyimide | a | b | c | d | [mmol/g] | weight [×10³] |
| C-101 | 100 | 0 | 100 | 0 | 0.00 | 101 |
| C-201 | 100 | 0 | 100 | 0 | 0.00 | 102 |
| C-301 | 40 | 60 | 70 | 30 | 0.00 | 88 |

### [Example 1] Preparation of Composite Membrane

<Preparation of Polyacrylonitrile (PAN) Porous Membrane Provided with Smooth Layer>

(Preparation of Radiation-Curable Polymer Containing Dialkylsiloxane Group)

39 g of UV9300 (photopolymerization initiator, manufactured by Momentive Performance Materials Inc.), 10 g of X-22-162C (manufactured by Shin-Etsu Chemical Co, Ltd.), and 0.007 g of DBU (1,8-diazabicyclo[5.4.0]undeca-7-ene) were added to a 150 mL three-neck flask and dissolved in 50 g of n-heptane. The state of the solution was maintained at 95° for 168 hours, thereby obtaining a radiation-curable polymer solution (viscosity at 25° C. was 22.8 mPa·s) containing a poly(siloxane) group.

(Preparation of Polymerizable Radiation-Curable Composition)

5 g of the obtained radiation-curable polymer solution was cooled to 20° C. and diluted with 95 g of n-heptane. 0.5 g of UV9380C (photopolymerization initiator, manufactured by Momentive Performance Materials Inc.) and 0.1 g of ORGATIX TA-10 (manufactured by Matsumoto Fine Chemical Co., Ltd.) were added to the obtained solution, thereby preparing a polymerizable radiation-curable composition.

(Coating of Porous Support with Polymerizable Radiation-Curable Composition and Formation of Smooth Layer)

The PAN porous membrane (membrane having the polyacrylonitrile porous membrane on the non-woven fabric, the membrane thickness including the non-woven fabric was approximately 180 μm) was used as a porous support and spin-coated with the polymerizable radiation-curable composition. Next, the membrane was subjected to a UV treatment (Light Hammer 10, D-valve, manufactured by Fusion UV System, Inc.) under conditions of a UV intensity of 24 kW/m for a treatment time of 10 seconds, and then the polymerizable radiation-curable composition was dried. In this manner, a smooth layer containing a dialkylsiloxane group and having a thickness of 1 μm was formed on the porous support.

<Preparation of Composite Membrane>

A gas separation composite membrane illustrated in FIG. 2 was prepared (a smooth layer is not illustrated in FIG. 2).

0.08 g of the polyimide (P-101) and 7.92 g of tetrahydrofuran were mixed in a 30 ml brown vial bottle and then stirred for 30 minutes, the PAN porous membrane to which the smooth layer was imparted was spin-coated with the mixture to form a gas separation layer, thereby obtaining a composite membrane. The thickness of the formed polyimide (P-101) layer was approximately 100 nm, and the thickness of the polyacrylonitrile porous membrane including the non-woven fabric was approximately 180 μm.

Further, a polyacrylonitrile porous membrane having a molecular weight cutoff of 100,000 or less was used. Further, the carbon dioxide permeability of the porous membrane at 40° C. and 5 MPa was 25000 GPU.

### [Examples 2 to 14] Preparation of Composite Membranes

Composite membranes were prepared in the same manner as in Example 1 except that the polyimide compound (P-101) was changed to each polyimide compound listed in Table 3 in the preparation of the composite membrane in Example 1.

### [Comparative Examples 1 to 3] Preparation of Composite Membranes

Composite membranes were prepared in the same manner as in Example 1 except that the polyimide compound (P-101) was changed to a polyimide compound (C-101), (C-201), or (C-301) in the preparation of the composite membrane in Example 1.

### Test Example 1

Measurement of toluene swelling ratio (weight change rate after exposure for 12 hours in toluene saturated atmosphere)

After each polyimide (0.2 g) and tetrahydrofuran (19.8 g) synthesized in the synthesis examples described above were mixed, the mixture was casted on a clean petri dish (10 cmϕ). The mixture was dried at a temperature of 25° C. for 12 hours and annealed at 90° C. for 7 days to prepare a polyimide single membrane (10 cmϕ, thickness of 20 μm), and the polyimide membrane was taken out from the petri dish. The weight of the obtained polyimide single membrane was measured, and the weight thereof after being exposed to a saturated toluene atmosphere was measured. More specifically, a 100 mL beaker was put into a metallic container which was covered by a toluene solvent and was able to be sealed by a lid, and the container was sealed by a lid and then allowed to stand for 12 hours. Subsequently, the polyimide single membrane was put into the beaker, the container was sealed by a lid and allowed to stand at 25° C. for 12 hours, the polyimide single membrane was taken out from the container, and then the weight thereof was measured.

The toluene swelling ratio was calculated according to the following equation.

Toluene swelling ratio (%)=100×{[weight (g) after exposure to toluene]−[weight (g) before exposure to toluene]}/[weight (g) before exposure to toluene]

### [Test Example 2] Evaluation of Gas Separation Performance

The gas separation performance of each gas separation composite membrane prepared in each example and each comparative example was evaluated based on the values (the gas permeability and the gas separation selectivity) obtained by performing measurement according to the following method.

Each gas separation composite membrane was used as a permeation test sample by cutting the entire porous support (support layer) such that the diameter of each membrane became 3 cm. These permeation test samples were placed in a SUS316 stainless steel cell (manufactured by DENISSEN Ltd.) having high pressure resistance, and the temperature of the cell was adjusted to 30° C. A mixed gas in which the volume ratio of carbon dioxide (CO₂) to methane (CH₄) was 10:90 was adjusted and supplied into the cell such that the total pressure on the gas supply side became 5 MPa (partial pressure of CO₂: 0.5 MPa) and the flow rate thereof became 130 mL/min. The gas permeabilities of CO₂ and CH₄ were measured using TCD (official name: Thermal Conductivity Detector) detection type gas chromatography. The gas permeabilities of the gas separation composite membranes prepared in each example and each comparative example were compared to each other by calculating gas permeation rates as the gas permeability (Permeance). The unit of the gas permeability (gas permeation rate) was expressed by the unit of GPU [1 GPU=1×10⁻⁶ cm³ (STP)/cm²·sec·cmHg]. The gas separation selectivity was calculated as the ratio (R_{CO2}/R_{CH4}) of the permeation rate R_{CH4} of CH₄ to the permeation rate R_{CO2} of CO₂ of the membrane.

The evaluation standard of the gas separation performance is as described below.
The gas separation composite membranes can be used without practical problems in a case where the gas separation performance was evaluated as AA to C.
AA: The gas permeability (R_{c02}) was 100 GPU or greater and the gas separation selectivity (R_{CO2}/R_{CH4}) was 20 or greater.
A: The gas permeability (R_{CO2}) was 80 GPU or greater and less than 100 GPU and the gas separation selectivity (R_{CO2}/R_{CH4}) was 20 or greater.
B: The gas permeability (R_{CO2}) was 50 GPU or greater and less than 80 GPU and the gas separation selectivity (R_{CO2}/R_{CH4}) was 20 or greater or the gas permeability (RCO₂) was 50 GPU or greater and the gas separation selectivity (R_{CO2}/R_{CH4}) was 15 or greater and less than 20.
C: The gas permeability (R_{CO2}) was less than 50 GPU and the gas separation selectivity (R_{CO2}/R_{CH4}) was 15 or greater or the gas separation selectivity (R_{CO2}/R_{CH4}) was 10 or greater and less than 15.
D: The gas separation selectivity was less than 10 or the test was not able to be carried out because the pressure was not applied.

### [Test Example 3] Evaluation of Gas Separation Performance after Exposure to Toluene

A 100 ml beaker was put into a metallic container which was covered by a toluene solvent and was able to be sealed by a lid, and the container was sealed by a lid and then allowed to stand for 12 hours. Subsequently, the permeation test sample of the gas separation composite membrane prepared in the same manner as in Test Example 2 was put into the beaker, the container was sealed by a lid and allowed to stand at a temperature of 25° C. for 10 minutes, and the sample was exposed to toluene. Next, the gas separation performance was evaluated in the same manner as in Test Example 2.

By exposing the sample to toluene, the plasticity resistance of the gas separation membrane with respect to impurities such as benzene, toluene, and xylene can be evaluated.

The results of each test example described above are listed in Table 3.

TABLE 3

| | | Copolymerization ratio | | | | | | | | |
|---|---|---|---|---|---|---|---|---|---|---|
| | | (molar ratio) | | | | | | Toluene | Gas separation | Gas separation |
| | | Acid | | | | | Weight-average | swelling | performance | performance |
| | | dianhydride | | Diamine | | Content A | molecular weight | ratio | (before exposure | (after exposure |
| | Polyimide | a | b | c | d | [mmol/g] | [×10³] | (%) | to toluene) | to toluene) |
| Example 1 | P-101 | 0 | 100 | 90 | 10 | 1.39 | 140 | 17 | A | A |
| Example 2 | P-102 | 100 | 0 | 100 | 0 | 3.31 | 103 | 13 | A | A |
| Example 3 | P-201 | 100 | 0 | 100 | 0 | 3.79 | 95 | 8 | AA | AA |
| Example 4 | P-202 | 0 | 100 | 100 | 0 | 1.52 | 110 | 12 | A | A |
| Example 5 | P-203 | 0 | 100 | 60 | 40 | 0.97 | 104 | 23 | A | B |
| Example 6 | P-204 | 0 | 100 | 20 | 80 | 0.35 | 77 | 37 | A | C |
| Example 7 | P-301 | 100 | 0 | 100 | 0 | 3.13 | 99 | 21 | A | B |
| Example 8 | P-302 | 100 | 0 | 0 | 100 | 1.97 | 131 | 26 | A | C |
| Example 9 | P-401 | 100 | 0 | 100 | 0 | 3.24 | 125 | 14 | A | A |
| Example 10 | P-402 | 50 | 50 | 50 | 50 | 1.70 | 122 | 22 | A | B |
| Example 11 | P-501 | 100 | 0 | 100 | 0 | 3.59 | 158 | 21 | A | B |
| Example 12 | P-502 | 50 | 50 | 50 | 50 | 1.79 | 196 | 36 | A | B |
| Example 13 | P-601 | 100 | 0 | 100 | 0 | 2.94 | 171 | 23 | A | B |
| Example 14 | P-602 | 50 | 50 | 50 | 50 | 1.61 | 85 | 37 | A | C |
| Comparative | C-101 | 100 | 0 | 100 | 0 | 0.00 | 101 | 42 | B | D |
| Example 1 | | | | | | | | | | |
| Comparative | C-201 | 100 | 0 | 100 | 0 | 0.00 | 102 | 41 | B | D |
| Example 2 | | | | | | | | | | |
| Comparative | C-301 | 40 | 60 | 70 | 30 | 0.00 | 88 | 45 | B | D |
| Example 3 | | | | | | | | | | |

The “content A” in Table 3 indicates the content (unit: mmol/g) of the structural portion represented by Formula (1) in the polyimide compound.

It was understood that each gas separation membrane of Example 1 to Example 14, obtained by using the polyimide compound having the structural portion represented by Formula (1) can achieve both of the gas permeability and the gas separation selectivity at high levels. Further, it was understood that these gas separation membranes were unlikely to be swollen even in a case of being exposed to a toluene atmosphere and excellent gas separation performance was able to be maintained.

On the contrary, it was found that each gas separation membrane of Comparative Examples 1 to 3, obtained by using the polyimide compound that did not have the structural portion represented by Formula (1) as in Comparative Example 1 to Comparative Example 3 had degraded gas separation performance, was easily swollen in a case of being exposed to a toluene atmosphere, and had degraded plasticity resistance.

Based on the comparison of the results of Example 3 to Example 6, it was understood that swelling of toluene was suppressed in a case where the amount of the structural portion represented by Formula (1), contained in the polyimide compound, was increased and the plasticity resistance was improved.

Based on the results of Examples 3, 9, 11, and 13, it was understood that there is a tendency that the polyimide compound having a structural portion which is a substituent (including a case of being a hydrogen atom) with less steric hindrance or a substituent having hydrogen bonding properties has excellent plasticity resistance and exhibits higher gas separation performance even in a case of being exposed to a toluene atmosphere, compared to the polyimide compound having a structural portion in which R^{3b}, R^{3g}, R^{4b}, and R^{4g} in Formulae (3-1) to (3-3) and Formulae (4-1) to (4-3) represent an alkyl group or an aryl group.

From the results described above, it was understood that a gas separation composite membrane having excellent gas separation performance and plasticity resistance, and a gas separation module and a gas separation method obtained by using the gas separation membrane of the present invention can be provided.

### EXPLANATION OF REFERENCES

1: gas separation layer
2: porous layer
3: non-woven fabric layer
10, 20: gas separation composite membrane

What is claimed is:

1. A gas separation membrane comprising:
a gas separation layer which contains a polyimide compound having a structural portion represented by Formula (1), 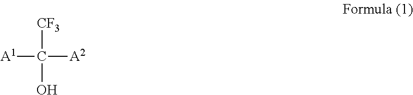
in Formula (1), A¹ and A² represent a linking site, a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, provided that at least one of A¹ or A² represents a linking site.

2. The gas separation membrane according to claim 1,
wherein the polyimide compound has a unit structure represented by Formula (2), 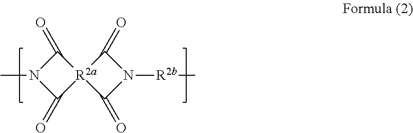
in Formula (2), R^{2a} represents a tetravalent linking group, and R^{2b} represents a divalent linking group, provided that at least one of R^{2a} or R^{2b} has the structural portion represented by Formula (1).

3. The gas separation membrane according to claim 2,
wherein R^{2a} in Formula (2) has the structural portion represented by Formula (1), and R^{2a} is represented by any of Formulae (3-1) to (3-3), 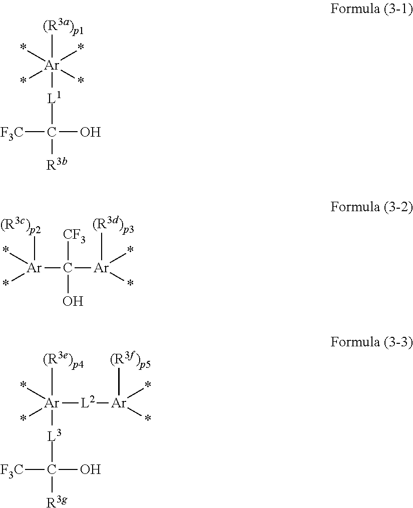
in Formulae (3-1) to (3-3), Ar represents an aromatic ring, the symbol “*” represents a linking site, L¹, L², and L³ represent a single bond or a divalent linking group, R^{3a}, R^{3c}, R^{3d}, R^{3e}, and R^{3f} represent a substituent, R^{3b} and R^{3g} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, and p1 to p5 represent an integer of 0 to 20.

4. The gas separation membrane according to claim 2,
wherein R^{2b} in Formula (2) has the structural portion represented by Formula (1), and R^{2b} is represented by any of Formulae (4-1) to (4-3), 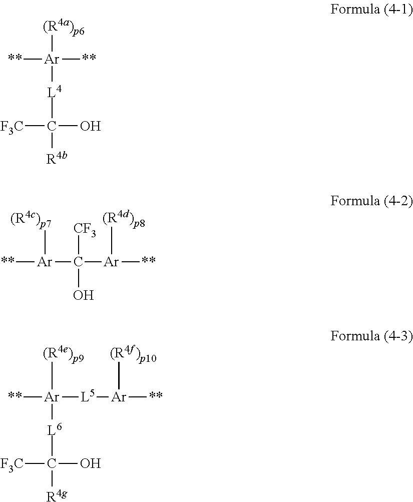
in Formulae (4-1) to (4-3), Ar represents an aromatic ring, the symbol “**” represents a linking site, L⁴, L⁵, and L⁶ represent a single bond or a divalent linking group, R^{4a}, R^{4c}, R^{4d}, R^{4e}, and R^{4f} represent a substituent, R^{4b} and R^{4g} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, and p6 to p10 represent an integer of 0 to 20.

5. The gas separation membrane according to claim 3,
wherein R^{2b} in Formula (2) has the structural portion represented by Formula (1), and R^{2b} is represented by any of Formulae (4-1) to (4-3), 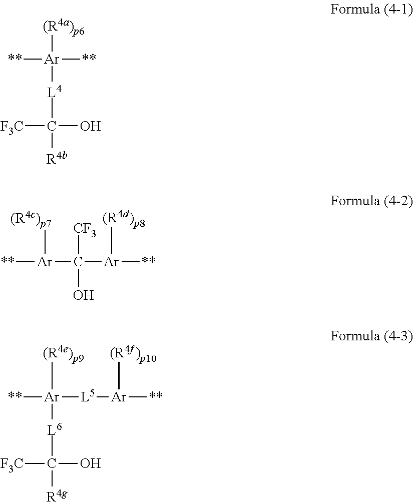
in Formulae (4-1) to (4-3), Ar represents an aromatic ring, the symbol “**” represents a linking site, L⁴, L⁵, and L⁶ represent a single bond or a divalent linking group, R^{4a}, R^{4c}, R^{4d}, R^{4e}, and R^{4f} represent a substituent, R^{4b} and R^{4g} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, and p6 to p10 represent an integer of 0 to 20.

6. The gas separation membrane according to claim 2,
wherein R^{ea} in Formula (2) is represented by any of Formulae (I-1) to (I-28), 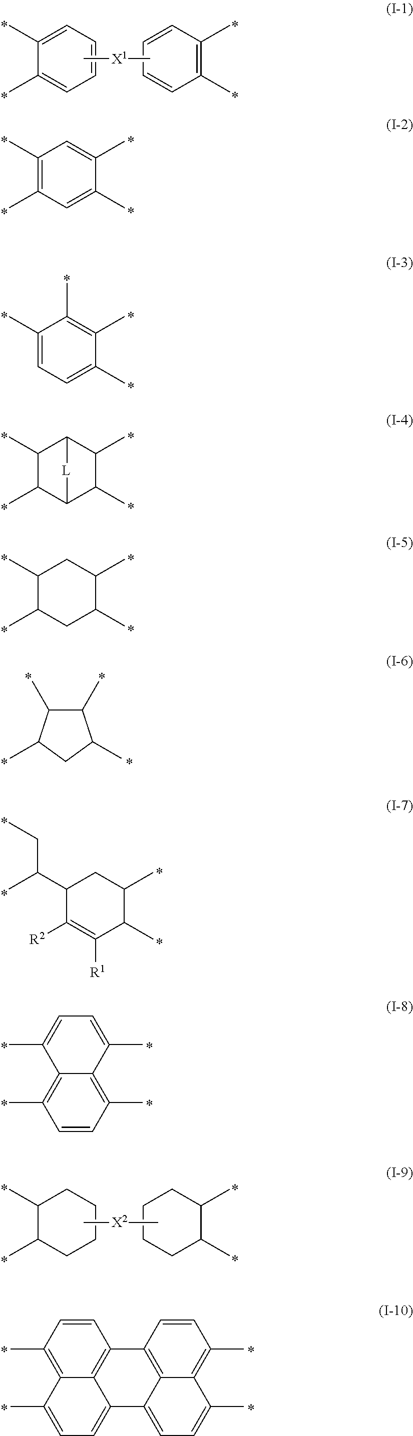 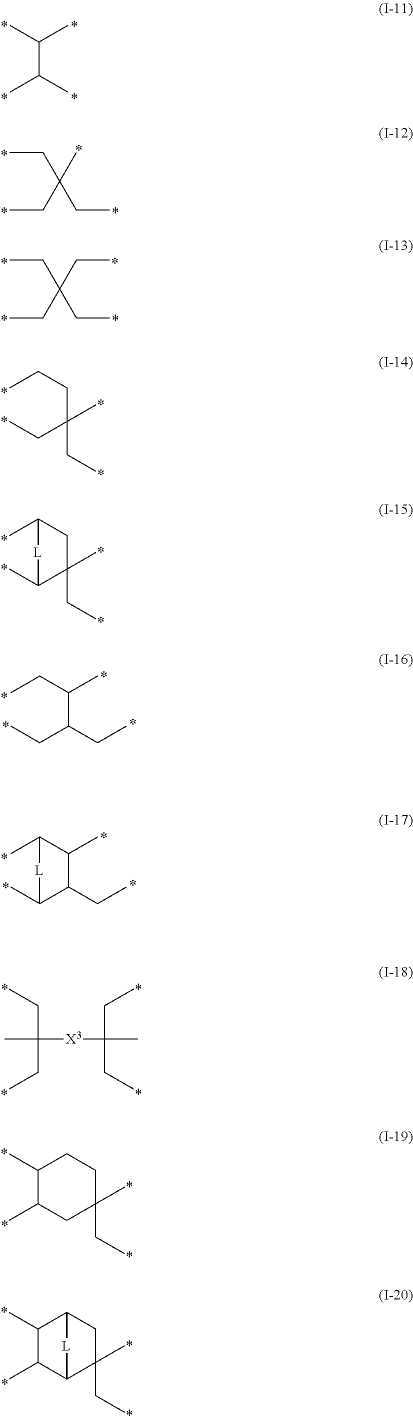 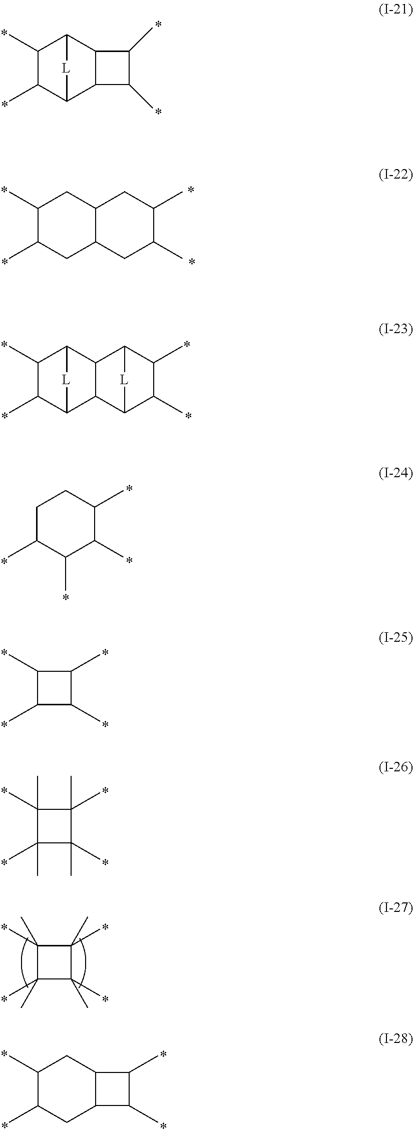
in Formulae (I-1) to (I-28), X¹ to X³ represent a single bond or a divalent linking group, L represents —CH═CH— or —CH₂—, R¹ and R² represent a hydrogen atom or a substituent that does not have a structural portion represented by Formula (1), and the symbol “*” represents a linking site.

7. The gas separation membrane according to claim 2,
wherein R^{2b} in Formula (2) is represented by Formula (II-a) or (II-b), 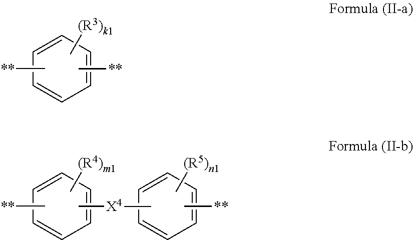
in Formula (II-a), R³ represents a substituent which does not have the structural portion represented by Formula (1), and k1 represents an integer of 0 to 4,
in Formula (II-b), R⁴ and R⁵ represent a substituent which does not have the structural portion represented by Formula (1) or groups that are linked to each other to form a ring together with X⁴, ml and n1 represent an integer of 0 to 4, and X⁴ represents a single bond or a divalent linking group, and
the symbol “**” represents a linking site.

8. The gas separation membrane according to claim 3
wherein R^{2b} in Formula (2) is represented by Formula (II-a) or (II-b), 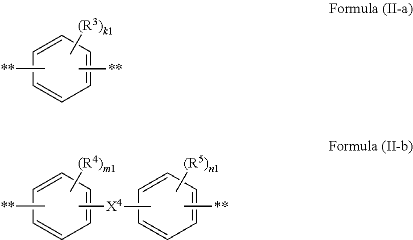
in Formula (II-a), R³ represents a substituent which does not have the structural portion represented by Formula (1), and k1 represents an integer of 0 to 4,
in Formula (II-b), R⁴ and R⁵ represent a substituent which does not have the structural portion represented by Formula (1) or groups that are linked to each other to form a ring together with X⁴, ml and n1 represent an integer of 0 to 4, and X⁴ represents a single bond or a divalent linking group, and
the symbol “**” represents a linking site.

9. The gas separation membrane according to claim 1,
wherein the content of the structural portion represented by Formula (1) in the polyimide compound is 0.50 mmol/g or greater.

10. The gas separation membrane according to claim 1,
wherein a toluene swelling ratio of the polyimide compound is 35% or less.

11. The gas separation membrane according to claim 1,
wherein the gas separation membrane is a gas separation composite membrane which includes a gas permeating support layer and the gas separation layer.

12. A gas separation module comprising:
the gas separation membrane according to claim 1.

13. A gas separator comprising:
the gas separation module according to claim 12.

14. A gas separation method which is performed using the gas separation membrane according to claim 1.

15. A polyimide compound which is represented by any of Formulae (5) to (7), 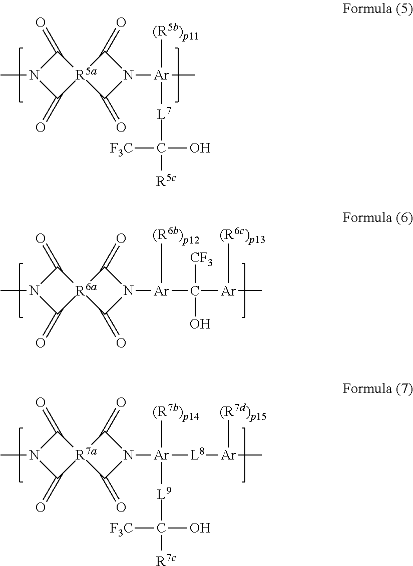
in Formulae (5) to (7), Ar represents an aromatic ring, R^{5a}, R^{6b}, and R^{7a} represent a tetravalent linking group, R^{5b}, R^{6b}, R^{6c}, R^{7b}, and R^{7d} represent a substituent, R^{5c} and R^{7c} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, L⁷, L⁸, and L⁹ represent a single bond or a divalent linking group, and p11 to p15 represent an integer of 0 to 20.

16. A polyimide compound which is represented by any of Formulae (8) to (10), 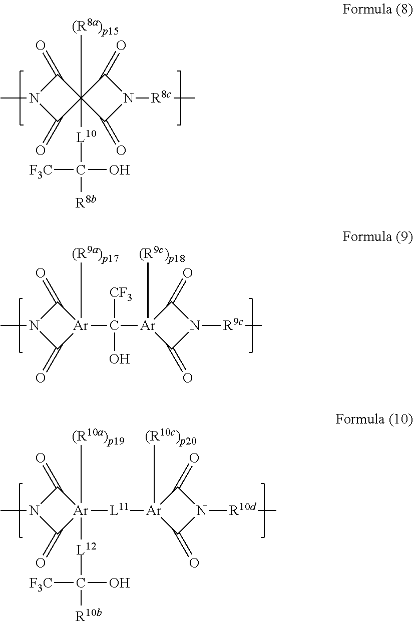
in Formulae (8) to (10), Ar represents an aromatic ring, R^{8a}, R^{9a}, R^{9b}, R^{10a}, and R^{10c} represent a substituent, R^{8b} and R^{10b} represent a hydrogen atom, a halogen atom, a carboxy group, a carbamoyl group, an acyl group, an acyloxy group, a sulfo group, a sulfamoyl group, an alkylsulfinyl group, an arylsulfinyl group, an alkylsulfonyloxy group, an alkoxycarbonyl group, a non-fluorinated alkyl group, or an aryl group, R^{8c}, R^{9c}, and R^{10d} represent a divalent linking group, L¹⁰, L¹¹, and L¹² represent a single bond or a divalent linking group, and p16 to p20 represent an integer of 0 to 20.
